(19) United States
(12) Patent Application Publication
(10) Pub. No.: US 2021/0353270 A1
(43) Pub. Date: Nov. 18, 2021

(54) TISSUE SAMPLE COLLECTION ASSEMBLY AND RELATED METHODS

(71) Applicants: Dallas ERDAHL, Fayetteville, AR (US); Logan MCDERMOT, Fayetteville, AR (US); Tomas BLODGETT, Fayetteville, AR (US); Shane SHELBOURN, Fayetteville, AR (US); Thomas RYAN, Fayetteville, AR (US); BioBotic Systems, Inc., Fayetteville, AR (US)

(72) Inventors: Dallas Erdahl, Fayetteville, AR (US); Logan McDermot, Fayetteville, AR (US); Tomas Blodgett, Fayetteville, AR (US); Shane Shelbourn, Fayetteville, AR (US); Thomas Ryan, Fayetteville, AR (US)

(21) Appl. No.: 17/280,751

(22) PCT Filed: Sep. 26, 2019
(86) PCT No.: PCT/US2019/053166

§ 371 Date: Mar. 26, 2021

Related U.S. Application Data
(60) Provisional application No. 62/738,810, filed on Sep. 28, 2018.

(51) Int. Cl. A61B 10/00; B01L 3/00

(52) U.S. Cl. CPC A61B 10/0096; B01L 3/52

(57) ABSTRACT

A tissue sample collection assembly includes a cap and an arm extending from an inner surface of the cap, a basket assembly including a top basket and bottom basket, and a bottle. The top basket is coupled with a second end of the arm. The basket assembly is disposed within the bottle, and the bottom basket is disposed at the bottom surface of the bottle. The arm and top basket have a first position within the bottle when the cap is sealed with the bottle, and the arm and top basket have a second position within the bottle when the cap is re-sealed with the bottle. The top basket is uncoupled from the bottom basket in the first position. The top basket is coupled with the bottom basket in the second position.

### RELATED APPLICATIONS

This application claims priority to U.S. provisional application No. 62/738,810 that was filed on 28 Sep. 2018, the entire content of which is hereby incorporated by reference herein.

### TECHNICAL FIELD

The present embodiments relate to a tissue sample collection assembly.

### BACKGROUND

Physicians often require biological tissue samples of patients to be tested to confirm or negate a diagnosis. The tissue samples are typically taken from the patient in one location, and then sent to another location for testing. The tissue samples must be properly preserved and stored for transportation to the testing location. A conventional way to store the tissue sample is with a container that has a reagent therein.

During use of a conventional tissue sample container, for example, similar to those shown in FIG. 60, where the sample container contains a reagent, a person taking a biopsy tissue sample places the sample into the container, and the tissue sample either drops off of the tool used to harvest the sample, or the tool is swished around in the formalin (reagent) to loosen the tissue sample from the tool, and drop the sample into the container. Then, the container is sent to the pathology lab for further processing and diagnosis of the tissue sample. At the pathology lab, a technician must manually open the sample container, remove the tissue sample from the container, and place the tissue sample into a conventional cassette, similar to the cassette shown in FIGS. 61-62.

Conventional sample containers introduce opportunities for human error and are time consuming to use. In addition, convention sample containers are currently insufficient to allow for automation of removal of the biopsy sample from the container for further processing.

### SUMMARY

A tissue sample collection assembly includes a cap and an arm extending from an inner surface of the cap, a basket assembly including a top basket and bottom basket, and a bottle. In one or more embodiments, the top basket includes one or more arm coupling members and the top basket is coupled with a second end of the arm. In one or more embodiments, the basket assembly is disposed within the bottle, and the bottom basket is disposed at or near the bottom surface of the bottle. The arm and top basket have a first position within the bottle when the cap is sealed with the bottle, and the arm and top basket have a second position within the bottle when the cap is sealed or re-sealed with the bottle. The top basket is uncoupled from the bottom basket in the first position. The top basket is coupled with the bottom basket in the second position.

In one or more embodiments, the basket assembly is rotatably coupled with the arm.

In one or more embodiments, the bottle has an open top portion, a bottom surface, a first set of side surfaces and a second set of side surfaces.

In one or more embodiments, the first set of side surfaces and the second set of side surfaces are planar.

In one or more embodiments, the first set of side surfaces and the second set of side surfaces are perpendicular to each other.

In one or more embodiments, the bottle further includes a guide disposed along at least one of the first set of side surfaces or the second set of side surfaces.

In one or more embodiments, the guide is a rail projecting inwardly along one or more of the first set of side surfaces or the second set of side surfaces.

In one or more embodiments, the basket assembly includes at least one basket guide member.

In one or more embodiments, at least one basket guide member is at least one recess configured to couple with the guide of the bottle.

In one or more embodiments, the tissue sample collection assembly includes one or more top basket top arm coupling members, and the arm comprising one or more basket coupling members that allow for the top basket to couple with the arm.

In one or more embodiments, the tissue sample collection assembly further includes one or more top basket bottom coupling members.

In one or more embodiments, the top basket is dome shaped to promote the migration of biopsy tissue samples suspended in the formalin (reagent) solution towards the center of the bottle, and thereby away from the outer walls of the bottle.

In one or more embodiments, the top basket has walls that extend downward. In one or more embodiments, the top basket has a chamfer on an inside lower edge of the top basket walls.

In one or more embodiments, the cap has a cap coupling member and the bottle has a bottle coupling member.

In one or more embodiments, the cap coupling member and the bottle coupling member is configured to couple and provide the arm/top basket a first position and a second position within the bottle. The top basket is uncoupled from the bottom basket in the first position. The top basket is coupled with the bottom basket in the second position. The arm/top basket sits deeper within the bottle in the second position compared to the first position. The arm/top basket is closer to the bottom basket or the bottom surface of the bottle in the second position compared to the first position.

In one or more embodiments, the cap has a side wall that has a cap coupling member. In one or more embodiments, the cap coupling member and/or the bottle coupling member has a two-stage or multistage coupling portion configured to provide the arm/top basket a first position and a second position within the bottle.

In one or more embodiments, the cap coupling member has a two-stage or multistage coupling portion that has a threaded portion (e.g., internally or externally threaded) and the bottle coupling member has a two-stage or multistage coupling portion that has a threaded portion (e.g., externally or internally threaded).

In one or more embodiments, the cap coupling member has a two-stage or multistage coupling portion that has a snap-fit coupling portion.

In one or more embodiments, the bottle coupling member has a two-stage or multistage coupling portion that has a snap-fit coupling portion.

In one or more embodiments, the cap coupling member has a pivoting coupling member. In one or more embodiments, the bottle coupling member has a two-stage or multi-stage coupling portion that has two projections to engage with the pivoting coupling member.

In one or more embodiments, a two-stage or multistage coupling portion has discrete coupling steps (e.g., two projections or snap-fit coupling portion), continuous coupling steps (e.g., threaded portion) or combination thereof to couple and provide the arm/top basket a first position and a second position within the bottle.

In one or more embodiments, a spacer is disposed at or near the bottle coupling member to prevent the arm/top basket from reaching the second position. In one or more embodiments, a spacer occupies one stage of a two-stage or multistage coupling portion to prevent the arm/top basket from reaching the second position. In one or more embodiments, a spacer occupies part of a threaded portion of a bottle coupling member to prevent the arm/top basket from reaching the second position.

In one or more embodiments, the cap is configured to provide the arm/top basket a first position and a second position within the bottle. In one or more embodiments, the arm is an integral part of the cap. In one or more embodiment, the arm is independent from the cap. In one or more embodiments, the cap is rotatably coupled with the arm. In one or more embodiment, the cap is configured to be pressed down to move the arm/top basket from a first position to a second position within the bottle.

The inventions also relate to methods of using or handling the tissue sample collection assembly described herein.

In one or more embodiments, the lab can use automation to further process the biopsy sample by using an automation assembly. The automation assembly includes part holding, dispensing, and manipulating fixtures, and a robot arm having a multi-purpose end-of-arm tool (gripper). The automation assembly further includes a supply column, and a camera.

In one or more embodiments, the gripper of the robot arm picks up the tissue sample collection assembly and places it in the fixture. The planar outer sides of the bottle mate within planar sides of the fixture, preventing the bottle from rotating while the cap is removed. The grip is used to remove the cap from the bottle, for example by rotating the cap relative to the bottle. The cap, arm and basket assembly are removed from the bottle, and held in view of the camera, which documents any patient indicia and/or the number, size, shape, color and types of tissue samples within the basket assembly. The robot arm moves the cap, arm and basket assembly over to a column of cassette frames, places the basket assembly within the cassette frame. The robot arm removes the arm from the top basket of the basket assembly and returns the arm and cap to the bottle for proper disposal. In one or more embodiments, the formalin is discarded from the bottle prior to placing the cap back on the bottle.

In one or more embodiments, the invention relates to a method of handling a tissue sample collection assembly comprising: 1) disposing a top basket of a basket assembly within a bottle of a tissue sample collection assembly, the tissue sample collection assembly comprising a cap having a side wall including a cap coupling member, the cap having an inner surface, an arm extending from a first end to a second end, the first end of the arm coupled with the inner surface of the cap; the basket assembly including the top basket and bottom basket; the top basket including one or more arm coupling members, the top basket coupled with the second end of the arm; the cap configured to be coupled with a bottle, the bottle having a bottle coupling member and an open top portion, the bottle having a bottom surface, a first set of side surfaces, and a second set of side surfaces; the bottom basket disposed at the bottom surface of the bottle; 2) coupling the cap with the bottle; and 3) moving the top basket to a first position within the bottle and sealing the cap with the bottle, where the top basket is uncoupled from the bottom basket in the first position.

In one or more embodiments, the method further includes disposing one or more biopsy samples within the bottle prior to moving the top basket from the first position to the second position.

In one or more embodiments, the method further includes moving the top basket from the first position to the second position and coupling the top basket with the bottom basket and sealing the cap with the bottle.

In one or more embodiments, moving the top basket from the first position to the second position includes rotating the cap relative to the bottle.

In one or more embodiments, the method further includes disposing a reagent within the bottle.

In one or more embodiments, the method further includes removing the cap, arm, and basket assembly from the bottle with an automation assembly.

In one or more embodiments, the invention relates to a method of handling a tissue sample collection assembly comprising 1) decoupling a cap and a bottle of the tissue sample collection assembly having a basket assembly within the bottle, the cap comprising an inner surface, an arm extending from a first end to a second end, the first end of the arm coupled with the inner surface of the cap, and a side wall including a cap coupling member; the basket assembly including a top basket and a bottom basket; the top basket including one or more arm coupling members, the top basket coupled with the second end of the arm; the cap configured to be coupled with the bottle, the bottle having a bottle coupling member, an open top portion, a bottom surface, a first set of side surfaces, and a second set of side surfaces; the bottom basket disposed at the bottom surface of the bottle; and 2) removing the cap, arm and the basket assembly from the bottle.

In one or more embodiments, decoupling includes rotating the cap relative to the bottle (e.g., with a gripper of an automation assembly).

In one or more embodiments, removing includes lifting the cap and the basket assembly from the bottle (e.g., with an automation assembly).

In one or more embodiment, the method further includes documenting the identity of the tissue sample collection assembly or documenting the tissue sample (e.g., with a camera of an automation assembly prior to, concurrent with or subsequent to decoupling and/or removing step(s)).

In one or more embodiment, the method further includes disposing the basket assembly within a cassette frame.

In one or more embodiment, the method further includes detaching the arm from the basket assembly (e.g., subsequent to disposing the basket assembly within a cassette frame).

In one or more embodiment, the method further includes discarding a reagent from the bottle.

### BRIEF DESCRIPTION OF THE DRAWINGS

In the detailed description of the embodiments presented below, reference is made to the accompanying drawings, in which:

FIG. 1 illustrates a perspective view of a tissue sample collection assembly in accordance with one or more embodiments.

FIG. 2 illustrates an exploded perspective view of a tissue sample collection assembly in accordance with one or more embodiments.

FIG. 3 illustrates a perspective view of a portion of a tissue sample collection assembly in accordance with one or more embodiments.

FIG. 4 illustrates a perspective view of a bottle of a tissue sample collection assembly in accordance with one or more embodiments.

FIG. 5A illustrates a perspective view of top basket of a tissue sample collection assembly in accordance with one or more embodiments.

FIG. 5B illustrates a perspective view of top basket of a tissue sample collection assembly in accordance with one or more embodiments.

FIG. 6 illustrates a perspective view of a bottom basket of a tissue sample collection assembly in accordance with one or more embodiments.

FIG. 7 illustrates a perspective view of a cassette frame of a tissue sample collection assembly in accordance with one or more embodiments.

FIG. 8 illustrates a cross-sectional view of a tissue sample collection assembly in the first position in accordance with one or more embodiments.

FIG. 9 illustrates a cross-sectional view of a tissue sample collection assembly in the second position in accordance with one or more embodiments.

FIG. 10 illustrates a perspective view of a tissue sample collection assembly in accordance with one or more embodiments.

FIG. 11 illustrates a cross-sectional perspective view of a portion of a tissue sample collection assembly in accordance with one or more embodiments.

FIG. 12 illustrates a cross-sectional perspective view of a tissue sample collection assembly in accordance with one or more embodiments.

FIG. 13 illustrates a cross-sectional perspective view of a tissue sample collection assembly in the cassette frame in accordance with one or more embodiments.

FIG. 14 illustrates a perspective view of an automation assembly in accordance with one or more embodiments.

FIG. 15 illustrates a perspective view of an automation assembly in accordance with one or more embodiments.

FIG. 16 illustrates a perspective view of a tissue sample collection assembly in accordance with one or more embodiments.

FIG. 17 illustrates a perspective view of a tissue sample collection assembly in accordance with one or more embodiments.

FIG. 18 illustrates a top view of a tissue sample collection assembly in accordance with one or more embodiments.

FIG. 19 illustrates a perspective view of a tissue sample collection assembly in accordance with one or more embodiments.

FIG. 20 illustrates a side view of a tissue sample collection assembly in accordance with one or more embodiments.

FIG. 21 illustrates a side view of a tissue sample collection assembly in accordance with one or more embodiments.

FIG. 22 illustrates a perspective view of a portion of a tissue sample collection assembly in accordance with one or more embodiments.

FIG. 23 illustrates a perspective view of a tissue sample collection assembly in accordance with one or more embodiments.

FIG. 24 illustrates a side view of a tissue sample collection assembly in accordance with one or more embodiments.

FIG. 25 illustrates a side view of a tissue sample collection assembly in accordance with one or more embodiments.

FIG. 26 illustrates a side view of a tissue sample collection assembly in accordance with one or more embodiments.

FIG. 27 illustrates a side view of a tissue sample collection assembly in accordance with one or more embodiments.

FIG. 28 illustrates a perspective view of a tissue sample collection assembly in accordance with one or more embodiments.

FIG. 29 illustrates a perspective view of a tissue sample collection assembly in accordance with one or more embodiments.

FIG. 30 illustrates a side view of a tissue sample collection assembly in accordance with one or more embodiments.

FIG. 31 illustrates a side view of a portion of a tissue sample collection assembly in accordance with one or more embodiments.

FIG. 32 illustrates a perspective view of a tissue sample collection assembly in accordance with one or more embodiments.

FIG. 33 illustrates a side view of a tissue sample collection assembly in accordance with one or more embodiments.

FIG. 34 illustrates a side view of a tissue sample collection assembly in accordance with one or more embodiments.

FIG. 35 illustrates a side view of a tissue sample collection assembly in accordance with one or more embodiments.

FIG. 36 illustrates a side view of a tissue sample collection assembly in accordance with one or more embodiments.

FIG. 37 illustrates a side view of a tissue sample collection assembly in accordance with one or more embodiments.

FIG. 38 illustrates a perspective view of a portion of a tissue sample collection assembly in accordance with one or more embodiments.

FIG. 39 illustrates a perspective view of a tissue transfer adapter plate in accordance with one or more embodiments.

FIG. 40 illustrates a perspective view of a tissue transfer adapter plate in accordance with one or more embodiments.

FIG. 41 illustrates a perspective view of a tissue sample collection assembly in accordance with one or more embodiments.

FIG. 42 illustrates a side view of a tissue sample collection assembly in accordance with one or more embodiments.

FIG. 43 illustrates a side view of a tissue sample collection assembly in accordance with one or more embodiments.

FIG. 44 illustrates a side view of a portion of a tissue sample collection assembly in accordance with one or more embodiments.

FIG. 45 illustrates a side view of a tissue sample collection assembly in accordance with one or more embodiments.

FIG. 46 illustrates a side view of a tissue sample collection assembly in accordance with one or more embodiments.

FIG. 47 illustrates a side view of a tissue sample collection assembly in accordance with one or more embodiments.

FIG. 48 illustrates a side view of a tissue sample collection assembly in accordance with one or more embodiments.

FIG. 49 illustrates a perspective view of a tissue sample collection assembly in accordance with one or more embodiments.

FIG. 50 illustrates a side view of a tissue sample collection assembly in accordance with one or more embodiments.

FIG. 51 illustrates a side view of a tissue sample collection assembly in accordance with one or more embodiments.

FIG. 52 illustrates a side view of a tissue sample collection assembly in accordance with one or more embodiments.

FIG. 53 illustrates a perspective view of a tissue sample collection assembly in accordance with one or more embodiments.

FIG. 54 illustrates a side cross-sectional view of a tissue sample collection assembly in accordance with one or more embodiments.

FIG. 55 illustrates a side cross-sectional view of a tissue sample collection assembly in accordance with one or more embodiments.

FIG. 56 illustrates a perspective view of a portion of a tissue sample collection assembly in accordance with one or more embodiments.

FIG. 57 illustrates a perspective view of a bottle in accordance with one or more embodiments.

FIG. 58 illustrates a perspective view of a bottom basket in accordance with one or more embodiments.

FIG. 59 illustrates a perspective view of a portion of a tissue sample collection assembly in accordance with one or more embodiments.

FIG. 60 illustrates a perspective view of a conventional tissue holders.

FIG. 61 illustrates a top view of a conventional cassette.

FIG. 62 illustrates a side perspective view of a conventional cassette.

The present embodiments are detailed below with reference to the listed figures.

### DETAILED DESCRIPTION OF THE EMBODIMENTS

In the following detailed description, reference is made to the accompanying drawings which form part of the description, and in which is shown by way of illustration specific embodiments in which the embodiments may be practiced. These embodiments are described in sufficient detail to enable those skilled in the art to practice the invention, and it is to be understood that other embodiments may be practiced in other ways. The following detailed description is not to be taken in a limiting sense, and the scope of the invention is defined by the detailed description, appended claims or their equivalents.

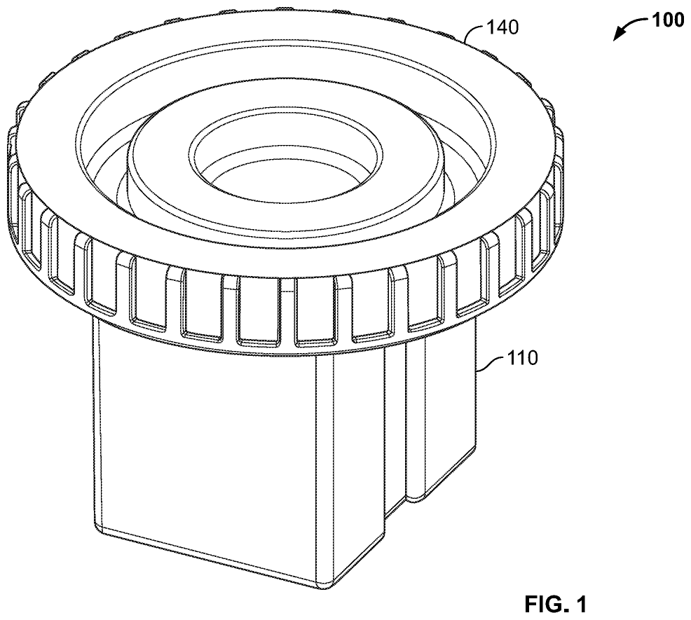

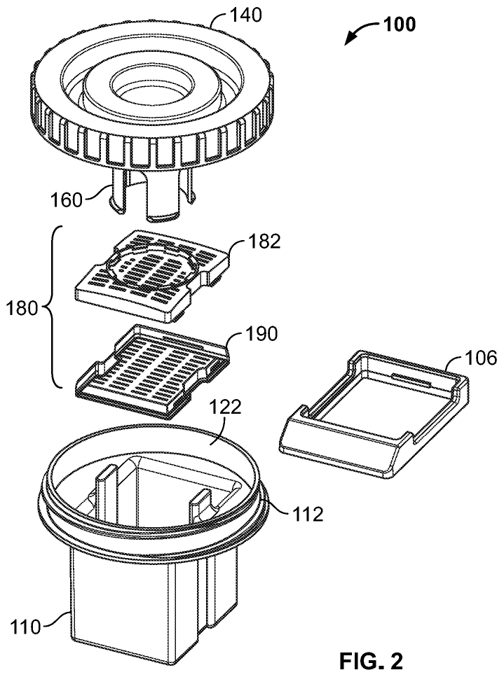

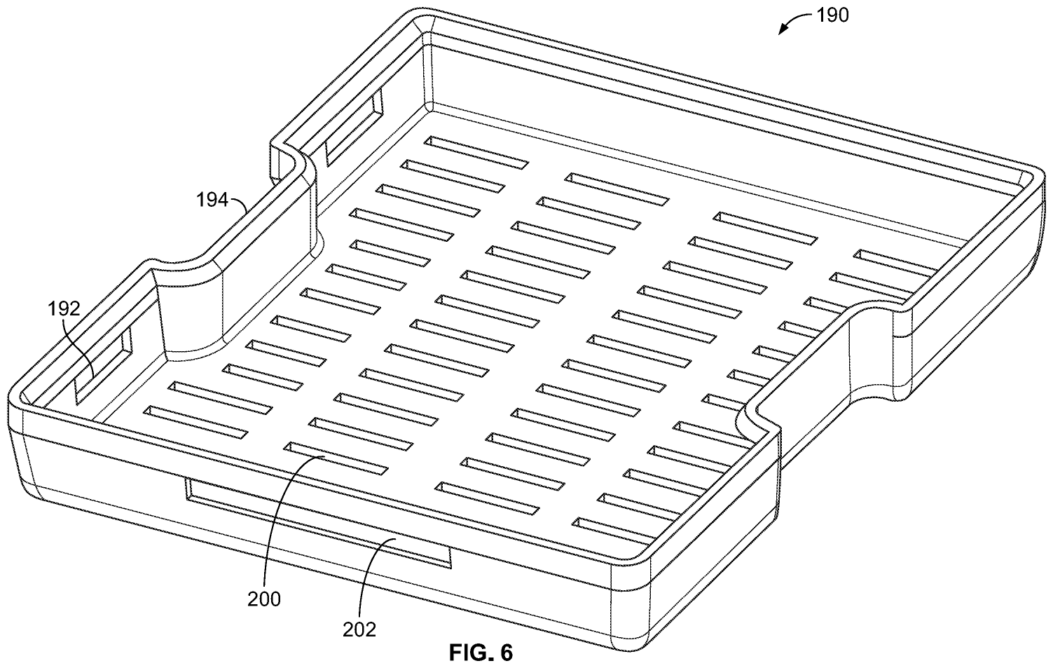

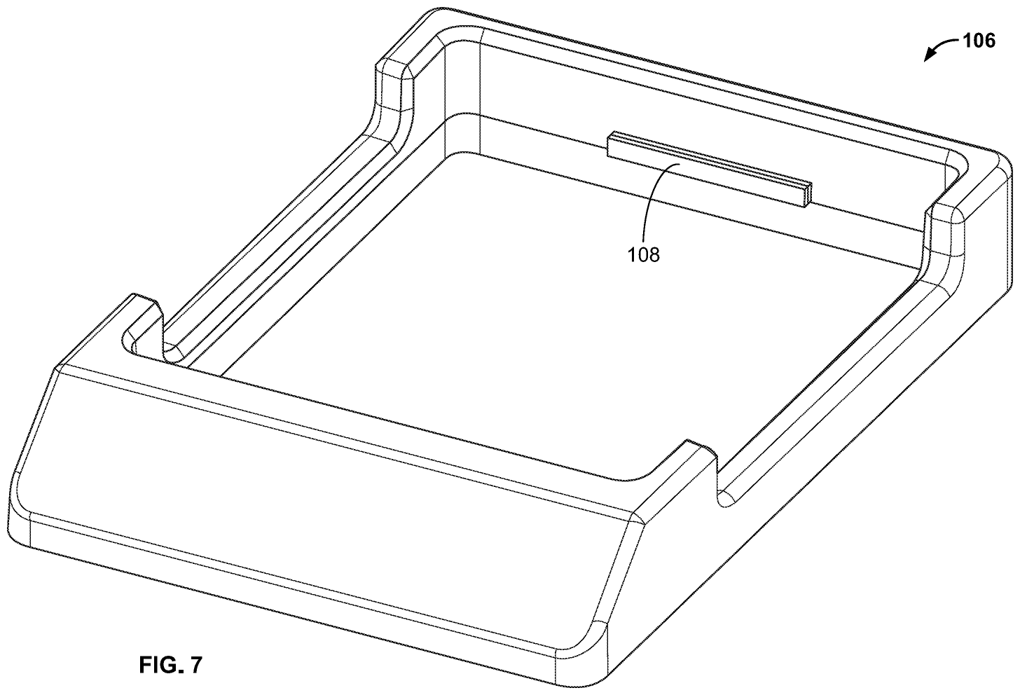

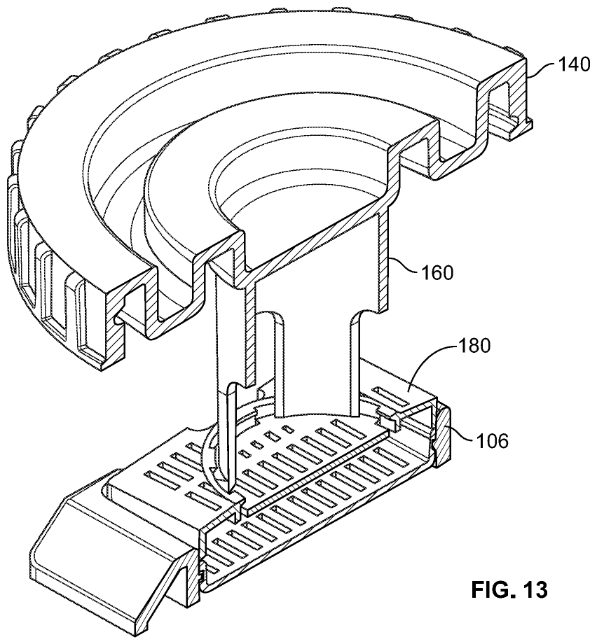

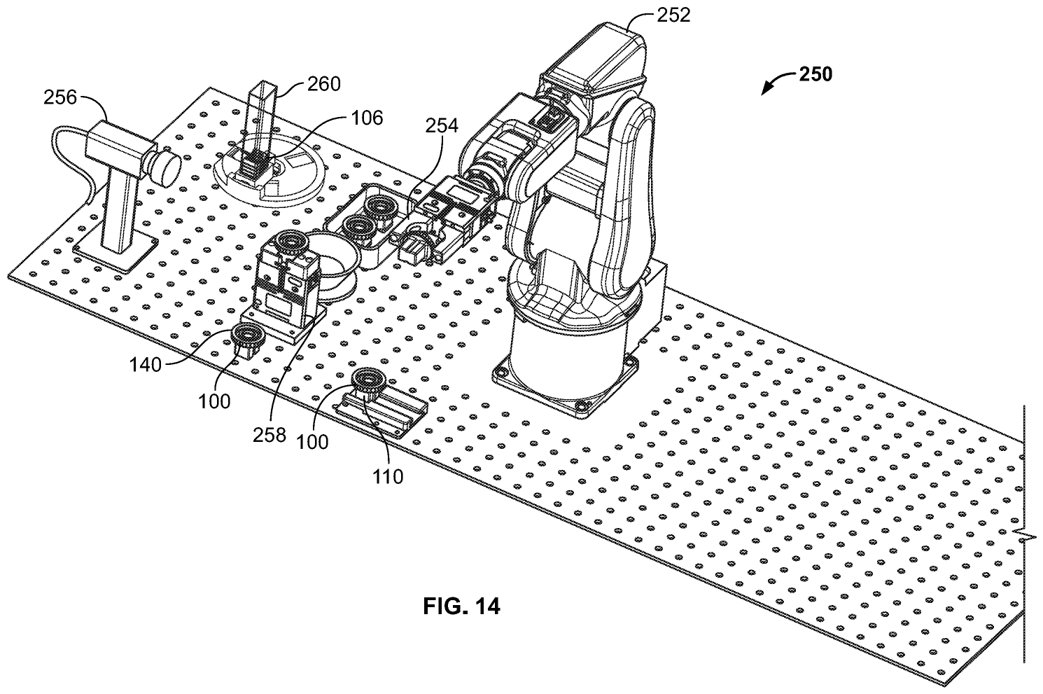

The present embodiments relate to a tissue sample collection assembly 100, as shown in in FIGS. 1 and 2. Generally, the tissue sample collection assembly 100 includes a cap 140, a bottle 110, an arm 160, and a basket assembly 180. The cap 140 seals with the bottle 110 to capture reagent disposed therein. The basket assembly 180 captures the biopsy tissue sample. The tissue sample collection assembly 100 and related automation assembly 250 (FIGS. 14-15) allow for the biopsy tissue sample to be conveniently collected within the bottle 110, and efficiently allows for further automated removal of the tissue sample from the bottle at the pathology lab, and allows for further automated processing of the biopsy. At the lab, the basket assembly 180 is disposed in and coupled with a cassette frame 106 (FIG. 7) for further processing. In one or more embodiments, the basket assembly 180 is snap-fitted with the cassette frame 106 (FIG. 13). In one or more embodiments, the cassette frame 106 includes one or more basket assembly coupling member 108 (FIG. 7). In one or more embodiment, the bottom basket 190 includes one or more cassette frame coupling member 202 (FIG. 6).

Referring to FIGS. 2-3, 8, the cap 140 includes a side wall 144 and a cap coupling member 142. In one or more embodiments, the cap coupling member 142 comprises a threaded portion 146, for instance an internally threaded portion. The cap 140 is further defined by an inner surface 148. In one or more embodiments, the cap 140 is coupled with the bottle 110. In one or more embodiments, the cap 140 is generally circular and has a disc-like shape. In one or more embodiments, the side wall 144 includes spaced-apart projections that allow for the cap 140 to be gripped with ease.

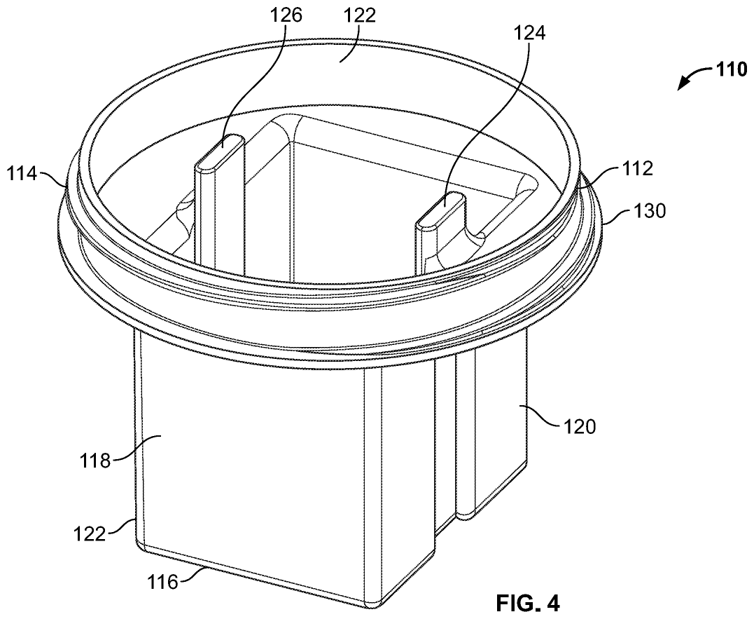

As shown in FIGS. 2, 4, and 8, the bottle 110 has an open top portion 122, and is defined in part by a bottom surface 116, a first set of side surfaces 118, and a second set of side surfaces 120. In one or more embodiments, the first set of side surfaces 118 and/or the second set of side surfaces 120 are planar. In one or more embodiments, the first set of side surfaces 118 and the second set of side surfaces 120 are perpendicular to each other. In one or more embodiments, the bottle 110 includes a guide 124 disposed along at least one of the first set of side surfaces 118 or the second set of side surfaces 120. In one or more embodiments, the guide 124 is a rail 126 that projects inwardly along one or more of the first set of side surfaces 118 or the second set of side surfaces 120. In one or more embodiments, the bottle 110 includes one, two, three, four, five or more guides 124 disposed along at least one of the first set of side surfaces 118 or the second set of side surfaces 120. In one embodiment, the bottle 110 includes one guide 124 disposed along one of the second set of side surfaces 120. In one embodiment, the bottle 110 includes two guides disposed along two of the second set of side surfaces 120 (FIG. 4).

The bottle 110 includes a bottle coupling member 112 that allows for the cap 140 to couple with the bottle 110. In one or more embodiments, the bottle coupling member comprises a threaded portion 114, for instance, an externally threaded portion. The cap 140 can be threadingly coupled with the bottle 110 to seal the cap 140 with the bottle.

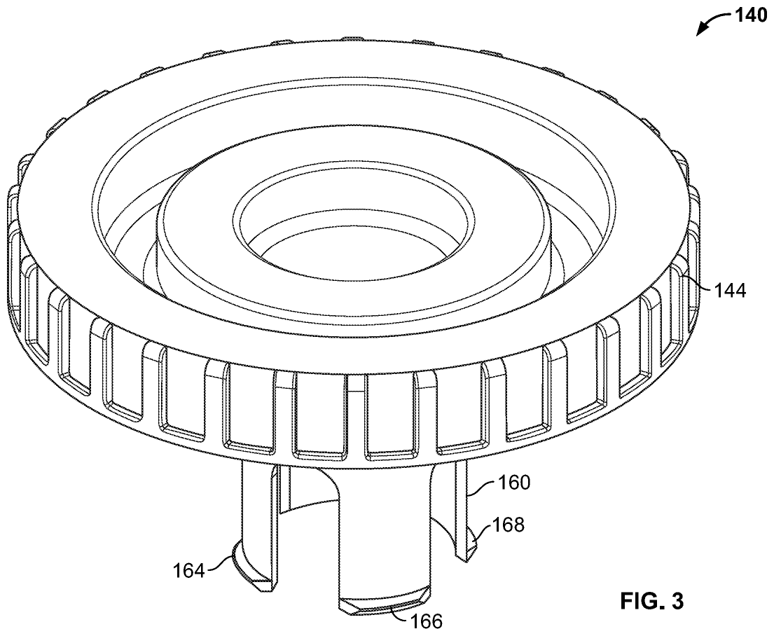

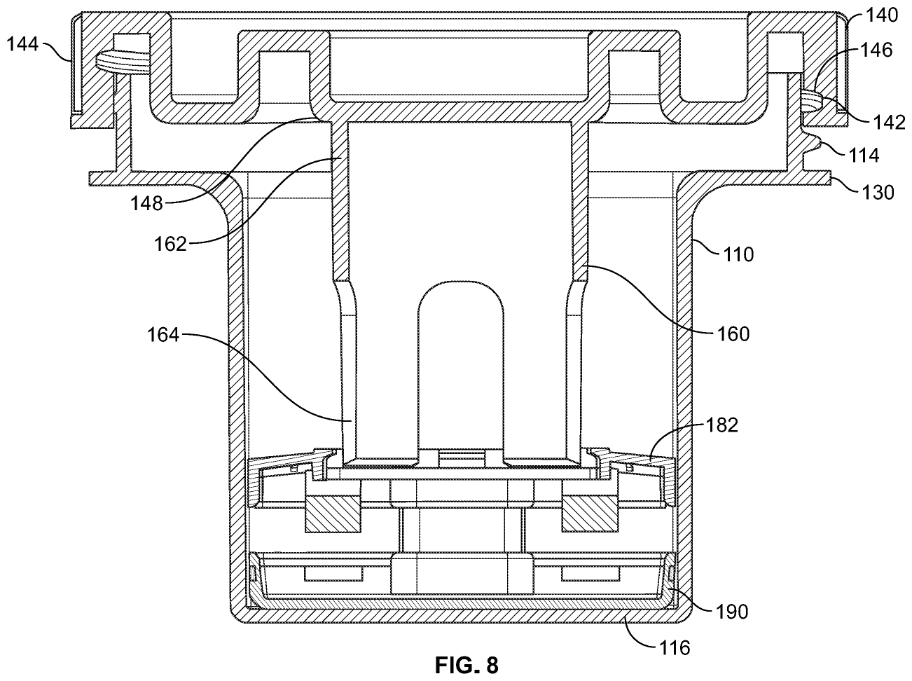

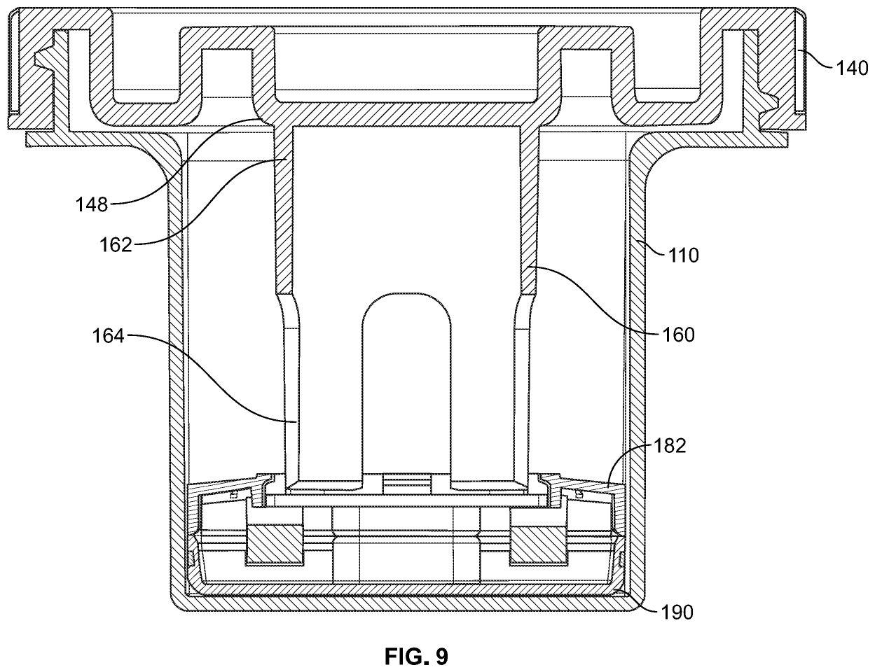

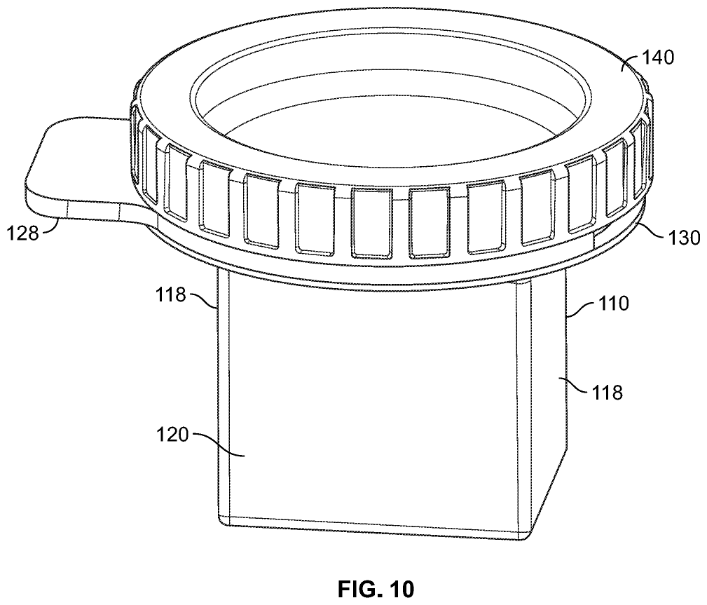

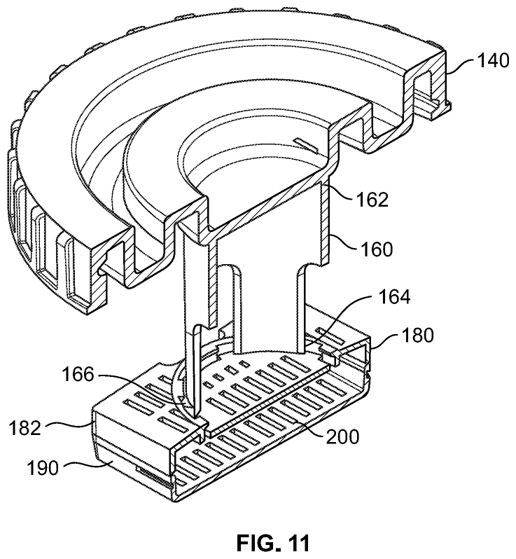

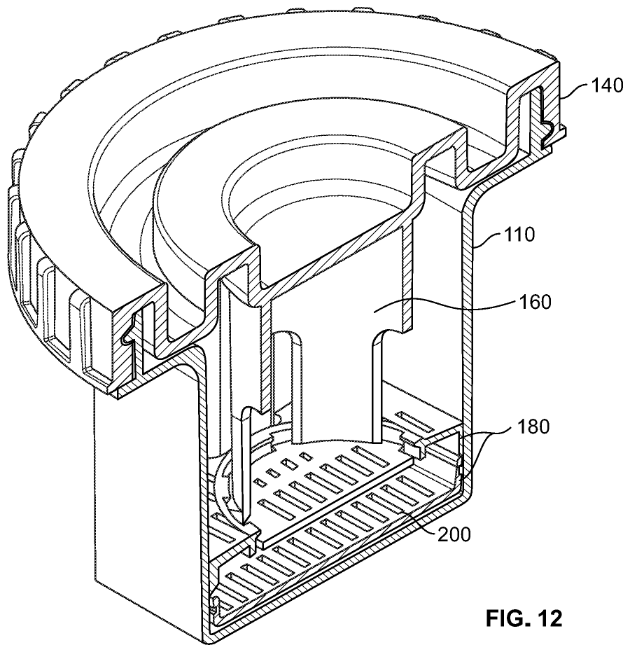

Disposed within the bottle 110 is an arm 160 and a basket assembly 180, as shown in FIGS. 8, 9, and 12. The arm 160 extends from a first end 162 to a second end 164, and the first end 162 is coupled with the inner surface 148 of the cap 140. The second end including at least one basket coupling member 166. In one or more embodiments, the at least one basket coupling member 166 includes one or more flanges 168 that extend from the second end 164 of the arm 160, as shown in FIGS. 3 and 11. The basket assembly 180 is coupled with the second end 164 of the arm 160.

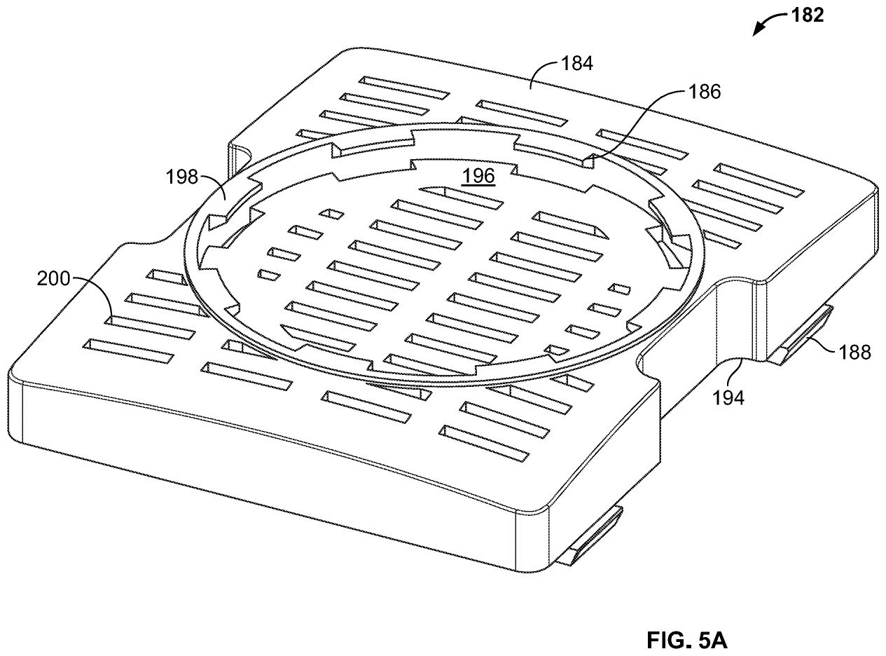

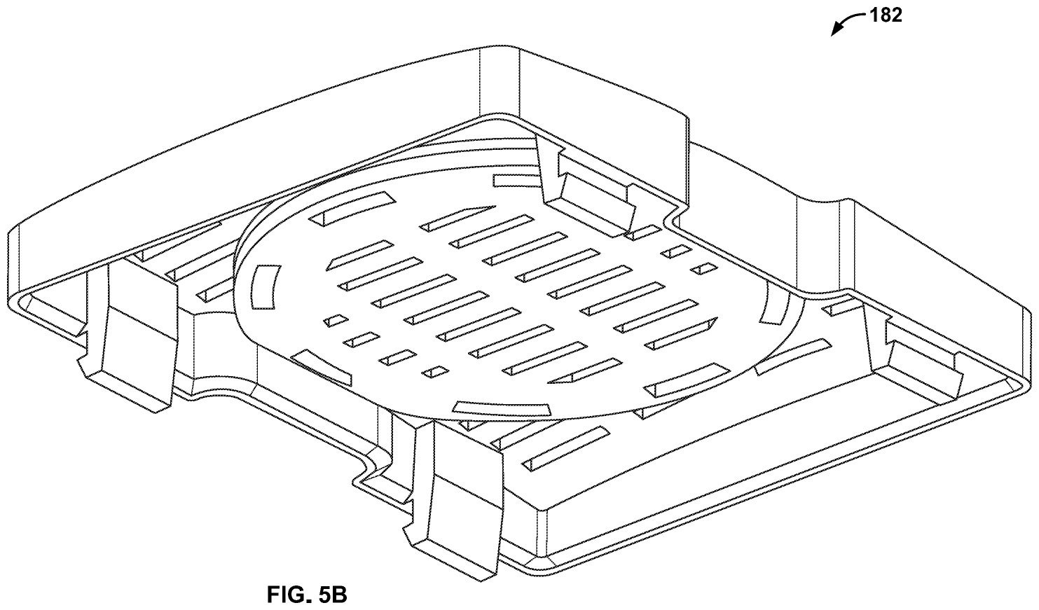

The basket assembly 180 including a top basket 182 (FIGS. 5A and 5B), and a bottom basket 190 (FIG. 6). The top basket 182 includes one or more top basket top arm coupling members 186 that allow for the top basket 182 to couple with the arm 160. In one or more embodiments, the top basket 182 includes a round opening 196 with one or more flanges 198 extending from within the round opening 196. The round opening 196, in combination with the round arm 160 allows for the basket assembly 180 to be rotatably coupled with the arm 160. The top basket 182 includes one or more top basket bottom coupling members 188 that allow for the top basket 182 to be coupled with the bottom basket 190. In one or more embodiments, the top basket bottom coupling members 188 include flanges that extend outward from a bottom and/or side surface of the top basket 182. The bottom basket 190 includes bottom basket coupling members 192 that allow for the bottom basket 190 to be coupled with the top basket 182. In one or more embodiments, the bottom basket coupling members 192 include one or more recesses within the bottom basket 190. In one or more embodiments, the basket assembly 180 include one or more fluid vents 200 that allow for fluid, such as a reagent within the bottle 110, can flow freely through the basket assembly 180.

In one or more embodiments, the top basket 182 includes a top basket top surface 184 that has a generally domed shape, with walls of the top basket 182 extending downward, and the chamfer on the inside lower edge of the walls, to promote tissue sample migration from the outer edges towards the center of the assembly during re-assembly immediately following initial tissue sample collection and placement into the bottle. The basket assembly 180, in one or more embodiments, has at least one planar side. In one or more embodiments, the basket assembly 180 includes a generally rectangular footprint, and four planar sides. As the cap 140 rotates, the arm 160 is able to rotate relative to the basket assembly 180, and the basket assembly 180 does not rotate relative to the bottle 110 when the cap 140 is rotated.

In one or more embodiments, the arm 160 and/or the top basket 182 have a first position (FIG. 8) within the bottle 110 while the cap 140 is sealed with the bottle 110. The arm 160 and/or the top basket 182 have a second position (FIG. 9) within the bottle 110 while the cap 140 is sealed with the bottle 110. In the first position, the top basket 182 is uncoupled from the bottom basket 190, as shown in FIG. 8. In the second position, the top basket 182 is coupled with the bottom basket 190, as shown in FIG. 9. In one or more embodiments, a spacer 128 is disposed between the cap 140 and a lip 130 of the bottle 110, as shown in FIG. 10. The spacer 128 is used to prevent the cap 140 from being placed in the second position.

In one or more embodiments, when the top basket 182 and/or bottom basket 190 are placed in the bottle 110, and the bottle 110 includes a guide 124, the top basket 182 and/or the bottom basket 190 include a basket guide member 194, and the basket guide member 194 slides along the guide of the bottle 110. In one or more embodiments, the basket guide member 194 is a recess that receives the rail 126 of the bottle 110 therein.

To use the tissue sample collection assembly 100, the bottom basket 190 is disposed on the bottom surface 116 of the bottle 110 and the bottle 110 is filled with a reagent. The arm 160 and top basket 182 are disposed within the bottle 110 as the cap 140 is coupled with the open top portion 122 of the bottle 110. The cap 140 is sealed with the bottle, for example by rotating and threading the cap 140 on to the bottle 110. As the cap 140 is rotated on to the bottle 110, the cap 140 rotates relative to the bottle 110, but the top basket 182 does not rotate relative to the bottle 110. The cap 140 is rotated until the cap 140 is sealed with the bottle 110 and the top basket 182 is placed in the first position, where the top basket 182 is not coupled with the bottom basket 190. At this point, the collection assembly 100 can be shipped to a clinic which harvests biopsy tissue.

When the tissue sample collection assembly 100 is at the clinic, the cap 140, arm 160, and top basket 182 (FIG. 11) are removed from the bottle 110 by twisting the cap 140 and removing from the bottle 110, and the bottom basket 190 remains in the bottom of the bottle 110. The biopsy sample can then easily be disposed within the bottom basket 190 by merely swishing the biopsy tool in the reagent and the biopsy sample falls off of the medical instrument and into the bottle 110, which is consistent with current practice in the clinic, and does not require the clinic to change current procedures. (This eliminates the extra step, as required in conventional designs of one having to open a collection basket after removing the cap, and also the biopsy tissue is more quickly placed in the reagent and has less exposure to air.) This step can be repeated multiple times as needed, until the clinic has determined that the optimal number of biopsy samples have been placed in the bottle 110. After the biopsy sample(s) has been placed in the bottle 110, some samples may have dropped into bottom basket 190, and some samples maybe floating in the reagent. The user can place the cap 140, arm 160, and top basket 182 back into the bottle 110 and capturing the biopsy sample between the top basket 182 and the bottom basket 190. As the top basket 182 is inserted into the bottle 110, the guide 124 of the bottle 110 guides the top basket 182 along the basket guide member 194 which keeps the top basket 182 generally parallel with the bottom basket 190. As the cap 140 is rotated relative to the bottle 110, the top basket 182 is moved to the first position, guiding any floating biopsy sample(s) into the basket assembly 180. The domed shape of the top basket 182, the parallel position of top basket 182, the walls of the top basket 182 extending downward, and the chamfer on the inside lower edge of the walls, ensure that all biopsy samples are guided into the basket assembly 180. As the cap 140 is further rotated, the top basket 182 is moved to the second position (FIG. 9), the top basket 182 is coupled with the bottom basket 190, and efficiently and securely encapsulating the biopsy sample(s) within the basket assembly 180. The cap 140 is also sealed with the bottle 110, and the biopsy sample can be shipped to the lab.

When the tissue sample collection assembly 100 is received at the pathology lab, the lab can use automation to further process the biopsy sample, as shown in FIGS. 14-15, by using an automation assembly 250. The automation assembly 250 includes a fixture 258, and a robot arm 252 having a grip 254. The automation assembly 250 further includes a supply column 260, and a camera 256.

The grip 254 of the robot arm 252 picks up the tissue sample collection assembly 100 and places it in the fixture 258. In one or more embodiments, the grip 254 includes structure that allows for the tissue sample collection assembly 100 to be picked up from the side and/or from the top. For example, grip 254 includes a rounded interior portion with flanges underneath to pick up the assembly 100 from the side. The planar outer sides of the bottle mate within planar sides of the fixture, preventing the bottle from rotating while the cap 140 is removed. The grip 254 is used to remove the cap 140 from the bottle 110, for example by rotating the cap 140 relative to the bottle 110. The cap 140, arm 160 and basket assembly 180 are removed from the bottle 110, and held in view of the camera 256, which documents any patient indicia and/or the number, size, shape, color and types of tissue samples within the basket assembly 180. The robot arm 252 moves the cap 140, arm 160 and basket assembly 180 over to a column of cassette frames 260, places the basket assembly 180 within the cassette frame 106. The robot arm 252 removes the arm 160 from the top basket 182 of the basket assembly 180 and returns the arm 160 and cap 140 to the bottle 110 for proper disposal. In one or more embodiments, the formalin reagent is discarded from the bottle 110 prior to placing the cap 140 back on the bottle 110. Any of the tissue sample collection assemblies herein can be used with the automation assembly 250 to further improve efficiencies of removing tissue samples from the tissue sample collection and to reduce human error.

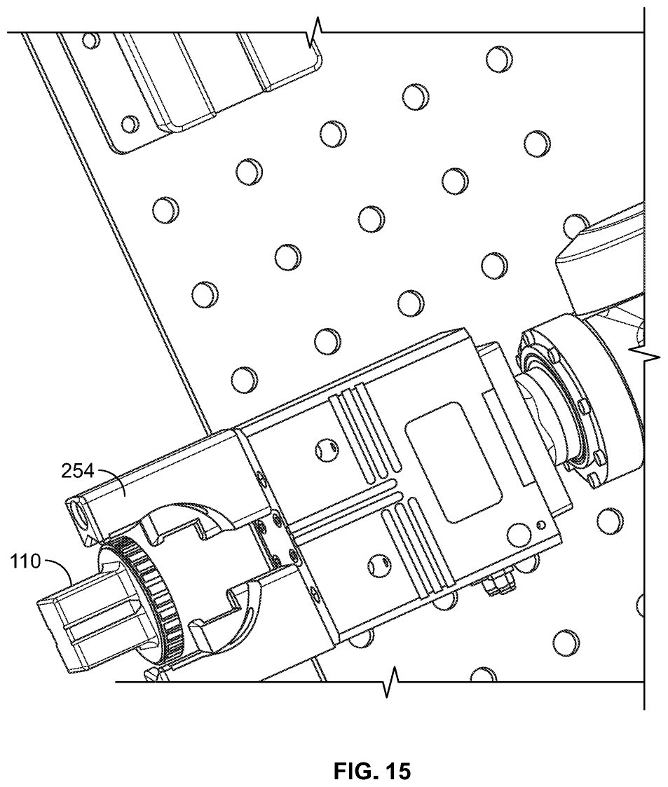

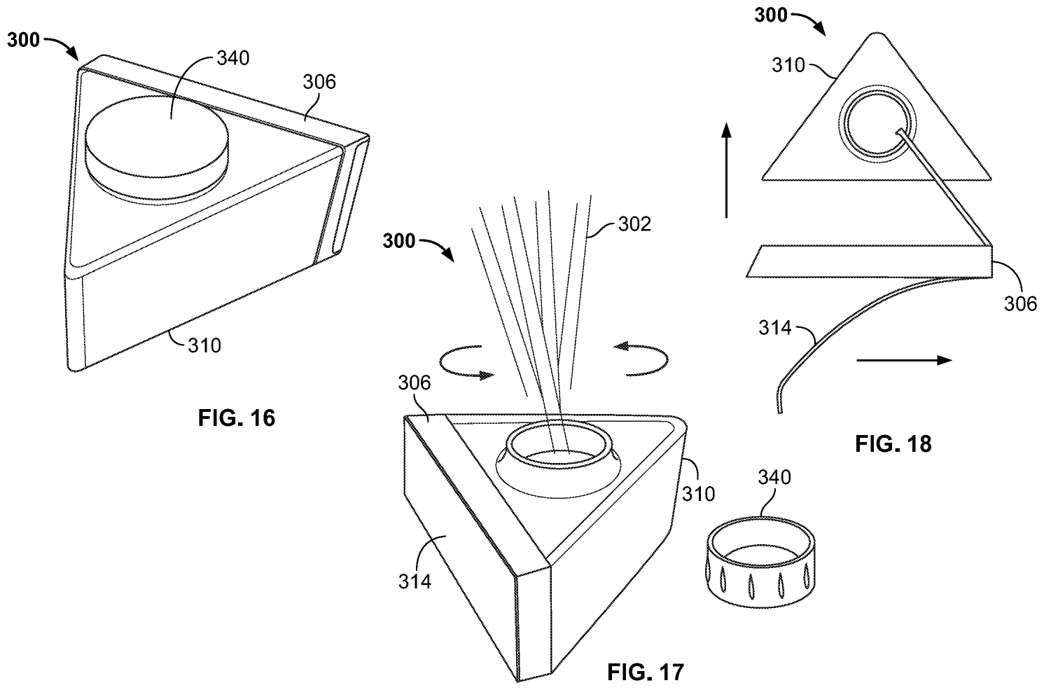

FIGS. 16-18 illustrate an additional embodiment for a tissue sample collection assembly 300. The tissue sample collection assembly 300 includes a bottle 310 and a cap 340. The bottle 310 includes a planar outer body, for example having an overall triangular shape. The tissue sample collection assembly 300 further includes a cassette 306. The cassette 306 has a size and shape to mate with one of the side surfaces of the bottle 310.

The cassette 306 is pressed and snapped into a side of the bottle 310. The cassette frame 306 and bottle 310 are encased by an adhesive seal 312 and then shrink wrapped for added leak prevention. The biopsy tool 302 is used (e.g., via swishing or dropping) to place the tissue sample into the bottle 310, the cap 340 is replaced and sealed to the bottle 310. The tissue sample collection assembly 300 is shipped to the lab for testing. At the lab, the shrink wrap is removed (e.g., peeled away) by a robotic arm, and the seal is removed (e.g., peeled away) by a robotic arm to drain the formalin and separate the cassette 306 from the bottle 310. The tissue sample is ready for further processing.

FIGS. 19-22 illustrate another embodiment of the tissue sample collection assembly 300. The tissue sample collection assembly 300 includes a rectangular shaped bottle 310 that is configured to receive and retain reagent 304 therein. The tissue sample collection assembly 300 further includes a silicon plug 341 and a cap 340, where the silicon plug 341 is configured and shaped to seal against the bottle 310 and retain the reagent 304 within the bottle 310. The cap 340 provides an additional seal. The tissue sample collection assembly 300 further includes a basket 390 for receiving tissue samples therein. The basket 390 is shaped and sized to be received in the bottom of the bottle 310. The tissue can be disposed directly into the basket 390 in the bottle 310 without having to first withdraw the basket 390 from the bottle 310.

After the tissue sample collection assembly 300 is sent to a clinic where the biopsy is being taken, the cap 340 and the plug are removed from the bottle 310, as shown in FIG. 20, and the tissue sample is disposed therein. The cap 340 and plug 341 are re-attached to the bottle 310. The tissue sample collection assembly 300 is sent to the lab, and the cap 340 and plug 341 are again removed by a robot arm. A robot arm 352 is used to place a cover on the basket, and to remove the covered basket 390 from the bottle 310, as shown in FIG. 21. A camera 356 is included to document the tissue samples as part of the testing process. Once removed from the reagent 304 and the bottle 310, the basket 390 with the tissue sample is disposed directly within a cassette frame 306, as shown in FIG. 22. The cassette frame 306 is used for further processing of the tissue sample.

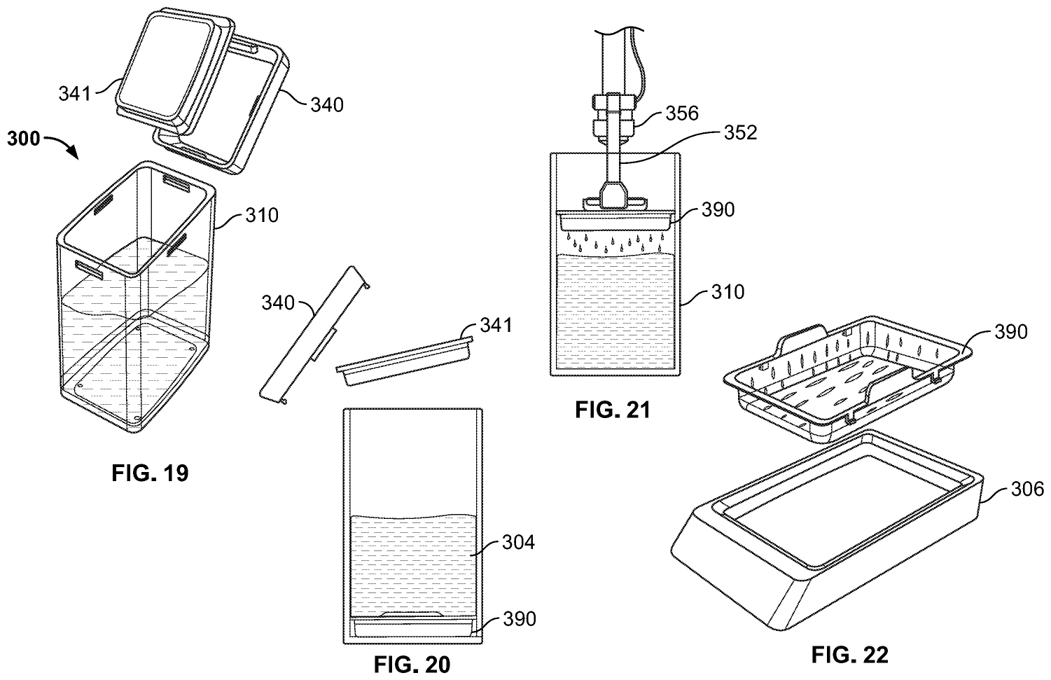

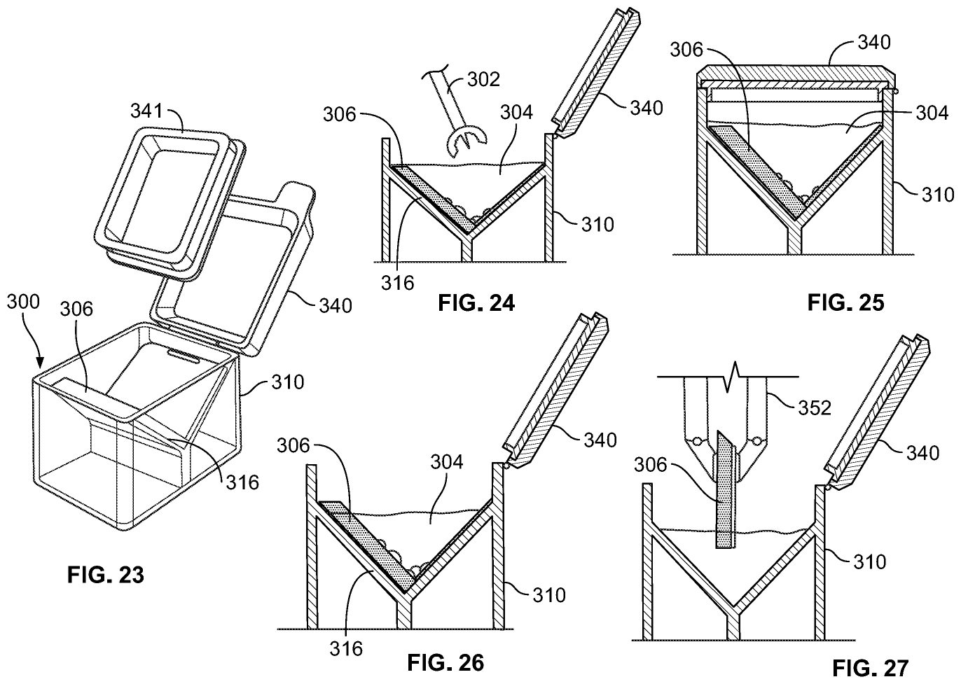

Referring to FIGS. 23-27, which illustrate another embodiment of the tissue sample collection assembly 300. The tissue sample collection assembly 300 includes a bottle 310 that is configured to receive and retain reagent 304 therein, and an angled bottom surface 316. The tissue sample collection assembly 300 further includes a silicon plug 341 and a hinged cap 340, where the silicon plug 341 is configured and shaped to seal against the bottle 310 and retain the reagent 304 within the bottle 310. The cap 340 provides an additional seal. The tissue sample collection assembly 300 further includes a cassette 306 for receiving tissue samples therein. The cassette 306 is shaped and sized to be received in on the angled bottom surface 316 of the bottle 310 and is preloaded in the bottle 310. The tissue can be disposed directly into the cassette 306 in the bottle 310 without having to first withdraw the cassette 306 from the bottle 310.

When the tissue sample collection assembly 300 is sent to a clinic where the biopsy is being taken, the cap 340 and the plug 341 are removed, as shown in FIG. 24, and the tissue sample is disposed therein with the biopsy tool 302. The cap 340 and the plug 341 are reinserted into and/or on the bottle 310, as shown in FIG. 25. The tissue sample collection assembly 300 is sent to the lab, and the cap 340 and plug 341 are again removed, as shown in FIG. 26. A robot arm 352 is used to close and remove the cassette 306 from the bottle 310, as shown in FIG. 27.

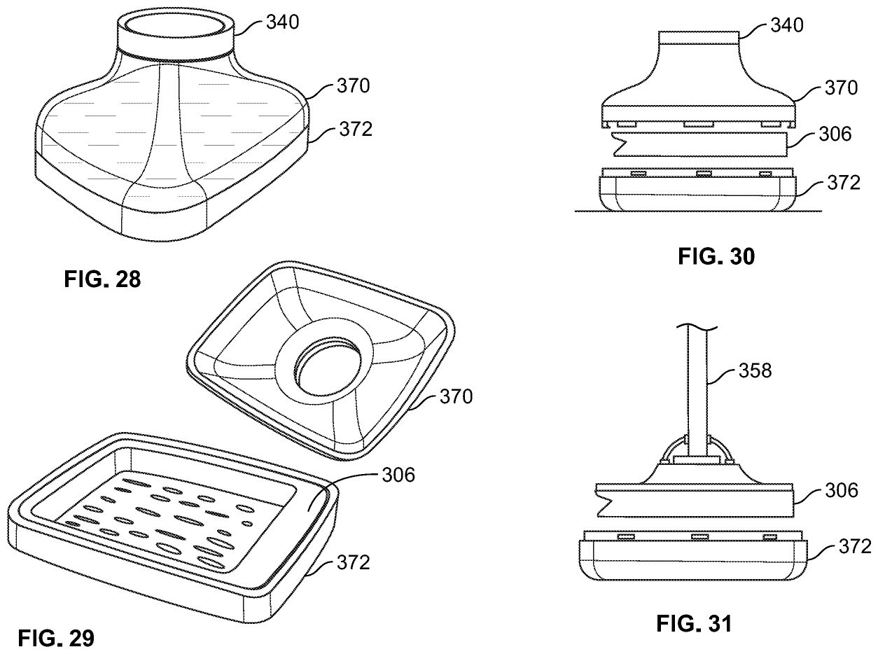

FIGS. 28-31 illustrate another embodiment of the tissue sample collection assembly 300. The tissue sample collection assembly 300 includes a two-part bottle with an upper housing 370 and a lower housing 372 that are coupled together, for example, with a snap-fit connection. The tissue sample collection assembly 300 further includes a cap 340 that couples with an opening in the upper housing 370, and seals the opening within the upper housing 370. A cassette 306 is pre-loaded in the lower housing along with a reagent in the lower housing 372 as shown in FIG. 29. The upper housing 370 is coupled with the lower housing 372, and the cap 340, such as a threaded cap, is coupled and sealed with the upper housing. In one or more embodiments, a shrink wrap is applied to the bottle to prevent fluid from leaking.

The bottle is sent to the clinic where the biopsy sample is taken from the patient. The cap 340 is removed and the tissue sample is placed through the opening in the upper housing 370 and into the cassette 306 in the lower housing 372. In one or more embodiments, the upper housing 370 is made of clear material so that the user can see the tissue samples being disposed in the cassette 306. The cap 340 is replaced on the bottle and the bottle is sent to the lab.

At the lab, the shrink wrap is removed by the robot arm and upper housing 370 is removed from the lower housing 372 by the robot arm. A suction transfer tool 358 attached to the robot arm is used to remove the cassette 306 with the tissue sample therein from the lower housing. The suction transfer tool 358 can also apply the cassette lid.

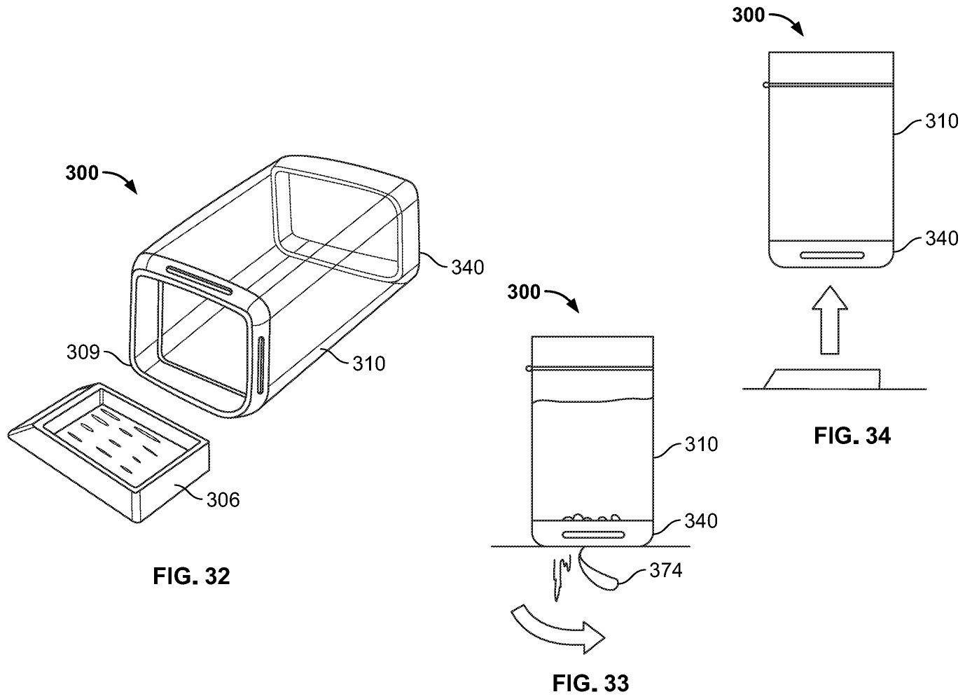

FIGS. 32-34 illustrate another embodiment of the tissue sample collection assembly 300. The tissue sample collection assembly 300 includes a bottle 310, a cap 340, and a cassette fixture 309. The cap 340 is coupled with the bottle 310 such that it is hinged with the bottle 310. A cassette 306 is placed into the bottom of the bottle 310, and an adhesive seal 374 is applied to the bottom of the cassette 306. The bottle is shrink wrapped for leak protection.

The cassette 306 is pre-loaded in the cassette fixture 309, reagent is disposed in the bottle 310, the cap 340 is sealed with the bottle 310, and the bottle 310 is shrink wrapped for lead prevention. The tissue sample collection assembly 300 is then sent to the clinic where the biopsy tissue sample is being taken. At the clinic, the cap 340 is opened, and the tissue sample is placed in the bottle 310 and disposed in the cassette 306. The cap 340 is re-sealed with the bottle 310 and the tissue sample collection assembly 300 is sent to the lab. At the lab, the shrink wrap is removed by the robot arm, the adhesive seal 374 is removed by the robot arm (FIG. 33), and the cassette 306 is removed from the bottle 310 by the robot arm directly lifting up the bottle 310, as shown in FIG. 34. The robot arm then places a cover on the cassette and the closed cassette is ready for further processing.

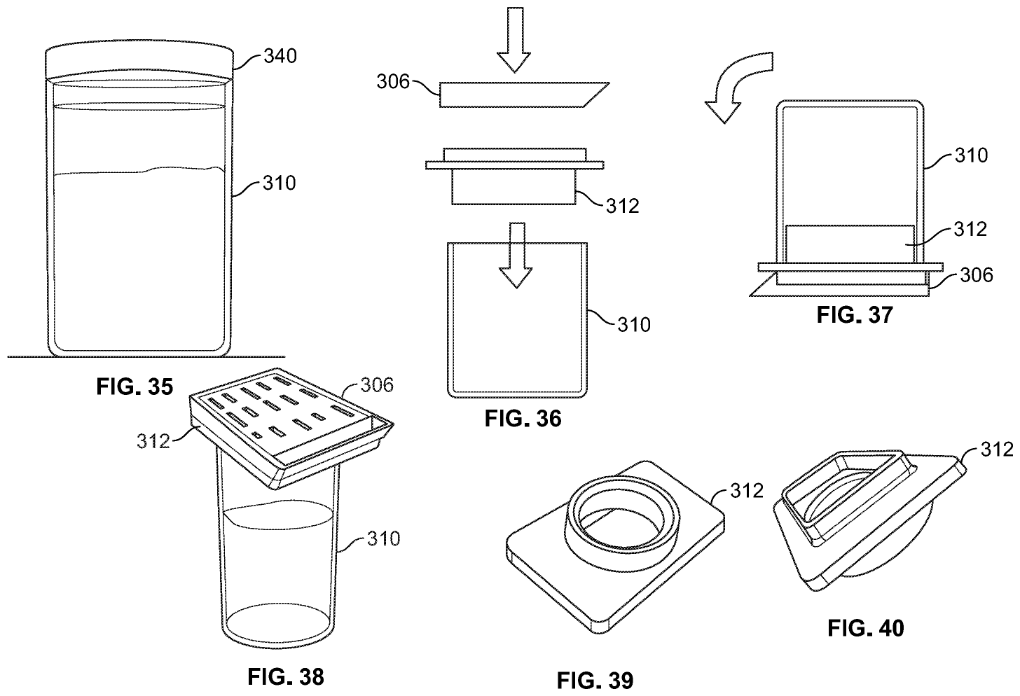

Referring to FIGS. 35-40, a tissue sample collection assembly 300 includes a bottle 310, cap 340, a cassette 306, and a tissue transfer adapter plate 312. The bottle 310 includes a reagent therein, and the cap 340 is sized and configured to seal the reagent, and the biopsy tissue sample, within the bottle 310. The tissue transfer adapter plate 312, shown in more detail in FIGS. 39-40, includes a bottle side (FIG. 39) and a cassette side (FIG. 40). The bottle side is sized and configured to seal with the bottle 310, for example with a circular shape. The cassette side is sized and configured to seal with the cassette, for example with a rectangular shape.

Once the sample is placed in the bottle 310 at the clinic, the bottle is sent to the lab. At the lab, the cap 340 is removed by the robot arm, and the tissue transfer adapter plate 312 is placed on the bottle 310 by the robot arm and sealed with the bottle 310. The cassette 306 is placed on and sealed with the tissue transfer adapter plate 312 (FIG. 36). Then, the bottle is rotated 180 degrees such that the cassette 306 is on the bottom and the bottle 310 is on the top (FIG. 37). This allows for the tissue sample to transfer to the cassette 306 and for the formalin to drain from the bottle 310 through holes in the cassette 306. These steps can be manually done, or a machine can automatically conduct all of these steps.

FIGS. 41-44 illustrate another embodiment of a tissue sample collection assembly 300. The assembly includes a bottle 310 and cap 340 as shown in FIG. 41, and a cassette 306 is preloaded into the bottle 310 prior to sending to the clinic for tissue sample collection. The bottle 310 has a rectangular interior shape to accommodate the rectangular shape of the cassette 306. A tissue diverter 334 is further coupled with the cassette 306.

After the tissue sample collection assembly 300 is sent to the lab for testing, a cap removal tool 332 removes the cap 340, and an optional camera can document the tissue sample. A formalin extractor 330 is used to remove the formalin from the bottle 310. For example, the formalin extractor 330 punctures the bottom of the bottle 310, as shown in FIG. 42, allowing the formalin to drain from the bottle 310. The floating tissue falls down into the cassette 306 and is guided in part by the tissue diverter 334.

As shown in FIG. 43, a suction transfer tool 358 is used to place a top lid on to the cassette 306 on the bottom surface of the bottle 310 and capturing the tissue sample within the cassette 306. The suction transfer tool 358 removes the cassette 306 with the tissue sample therein for further processing.

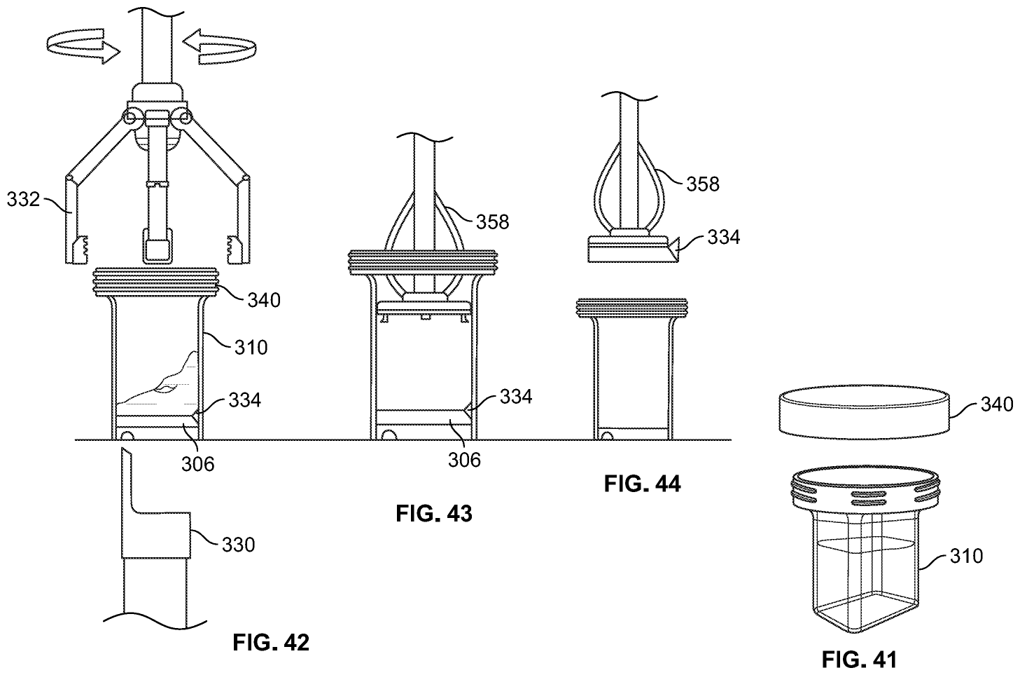

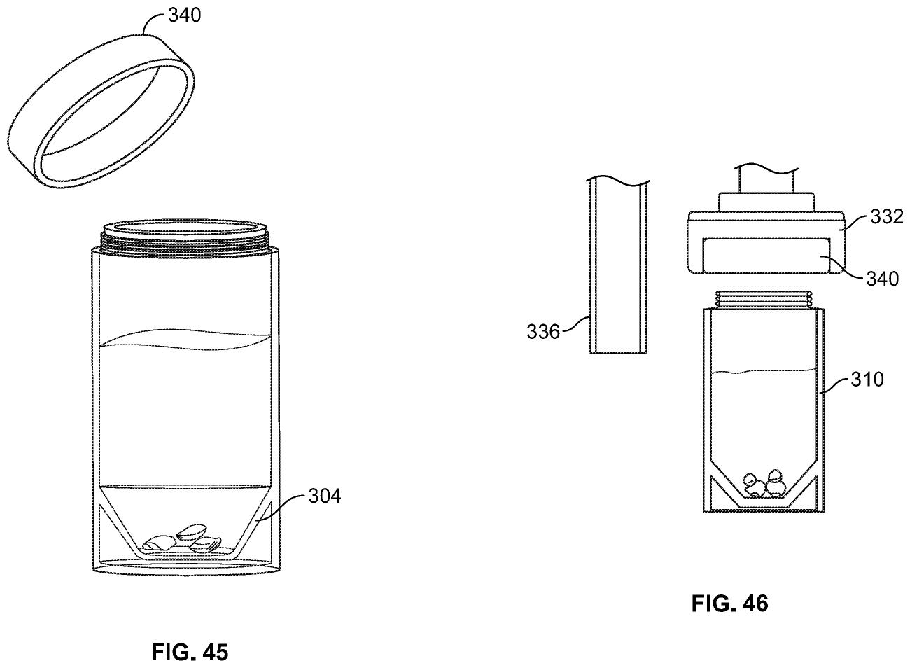

FIGS. 45-48 illustrate another embodiment of the tissue sample collection assembly 300. The tissue sample collection assembly 300 includes a cap 340 and a bottle 310. The bottle 310 includes a beveled bottom 304, as shown in FIG. 45. As a clinician places the tissue sample into the bottle 310, the beveled bottom 304 funnels the tissue samples into one location for easy removal using a pipette.

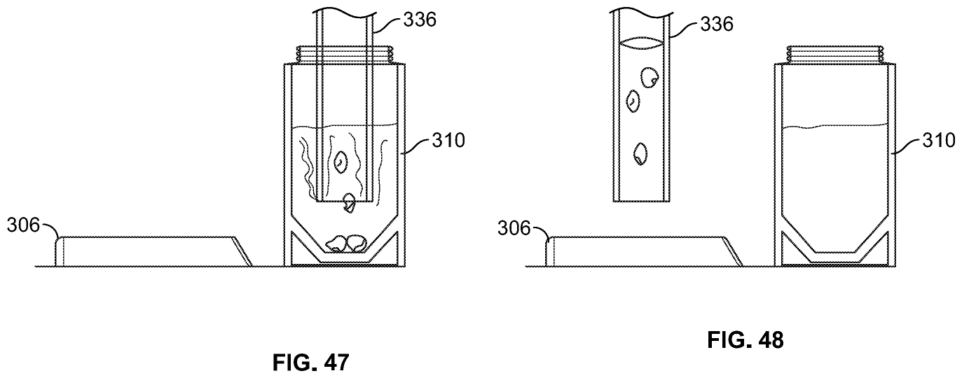

After tissue sample are placed into the bottle 310, the cap 340 is replaced, and the tissue sample collection assembly 300 is sent to the lab. At the lab a cap removal tool 332 is used to automatically remove the cap 340 (FIG. 46). A pipette 336 is disposed in the bottle 310, and the tissue samples and formalin are drawn from the bottle 310, as shown in FIG. 47. The pipette 336 is used to transfer the tissue samples and formalin to the cassette 306, where they are deposited within the cassette 306 and the formalin drains through the cassette 306 (FIG. 48).

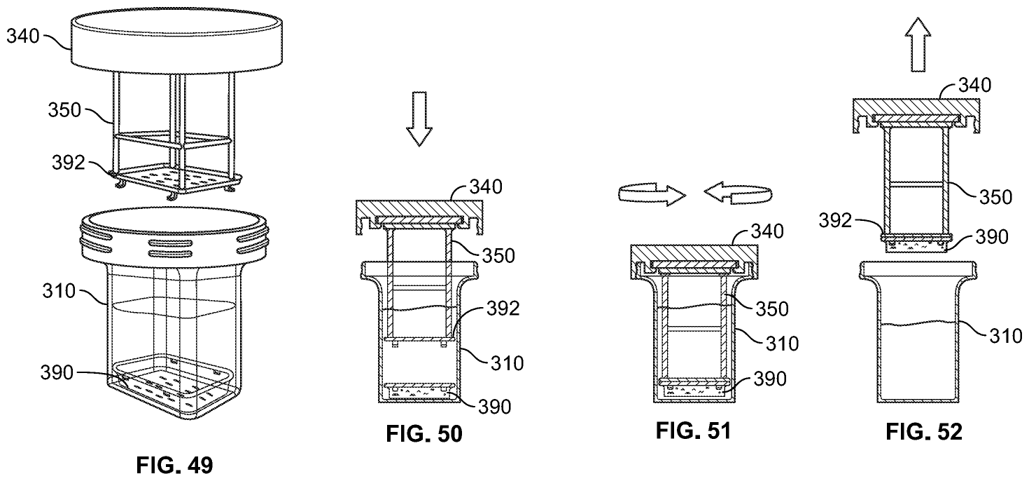

Referring to FIGS. 49-52, another embodiment of the tissue sample collection assembly 300 is shown. The tissue sample collection assembly 300 includes a cap 340, support structure 350, a bottle 310, a tissue basket 390, and a basket lid 392. The cap 340 is configured to seal with the bottle 310, for example with a threaded connection. The support structure 350 is coupled to the inner surface of the cap 340, for example, by one or more flanges that allow for the cap 340 to rotate relative to the support structure 350. The bottle 310 include planar sides, such as a rectangular inner portion that allows for a rectangular tissue basket 390 to be disposed therein. The tissue basket 390 and basket lid 392 include coupling features, such as snap-fit coupling features that allow for the tissue basket 390 and basket lid 392 to be coupled and uncoupled from one another. The support structure 350 is further coupled with the basket lid 392 (prior to the taking of the biopsy tissue sample), as shown in FIGS. 49-50.

The tissue sample collection assembly 300 is sent to the clinic where the biopsy sample is taken from the patient. The cap 340, support structure 350, and basket lid 392 are removed from the bottle 310, which has formalin therein. The user places the tissue sample within the basket 390 that is in the bottom of the bottle 310. The cap 340, support structure 350, and basket lid 392 are placed back in bottle 310, as shown in FIG. 50. The cap is threaded onto the bottle 310. Then the cap stops threading, the user presses down and turns to snap the basket lid 392 on to the basket 390 as shown in FIG. 51. Accordingly, the support structure 350 has a first position within the bottle when the cap is threaded on to the bottle 310, the basket lid 392 remained uncoupled from the tissue basket 390 in the first position. The support structure 350 has a second position within the bottle when the cap is threaded on to the bottle 310, the basket lid 392 is coupled with the tissue basket 390 in the second position. The cap is configured to be pressed down to move support structure 350/basket lid 392 from a first position to a second position within the bottle. The cap 340 is independent from the internal lid structure.

The tissue sample collection assembly 300 can be sent to the lab for further processing. At the lab, the cap 340 is removed from the bottle 310, along with the support structure 350 and the lidded basket 390. The tissue basket 390 is removed from the support structure, for example, by clipping the support structure 350 away from the basket lid 392.

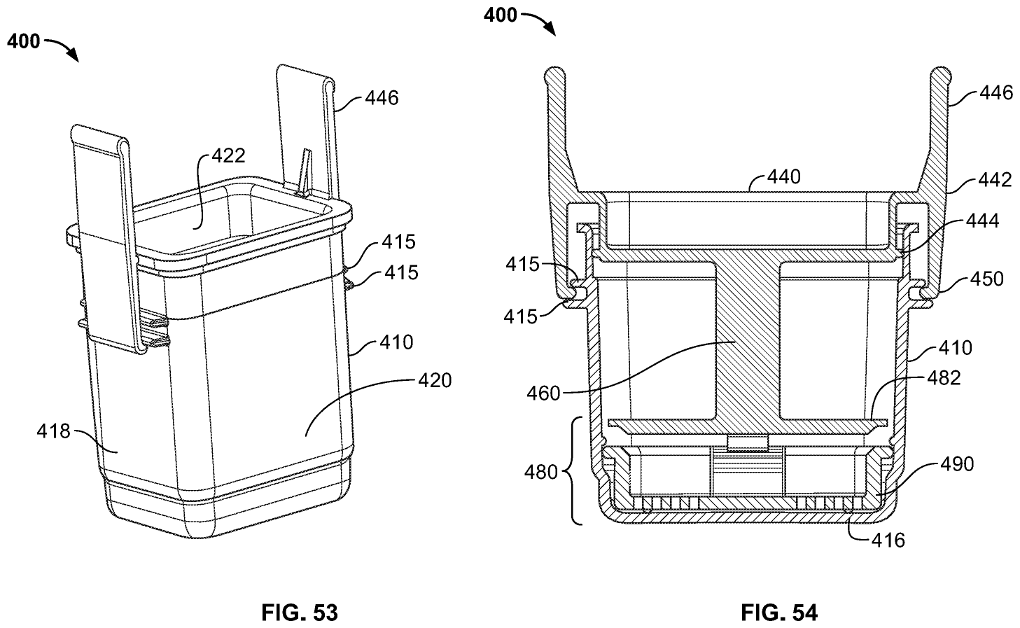

FIGS. 53-59 illustrate another embodiment of the tissue sample collection assembly 400. The tissue sample collection assembly 400 includes a cap 440, a bottle 410, an arm 460, and a basket assembly 480. The cap 440 seals with the bottle 410 to capture reagent disposed therein. The basket assembly 480 captures the biopsy tissue sample. The tissue sample collection assembly 400 and related automation assembly 250 (FIGS. 14-15) allow for the biopsy tissue sample to be conveniently collected within the bottle 410, and efficiently allows for further automated removal of the tissue sample from the bottle at the pathology lab, and allows for further automated processing of the biopsy. At the lab, the basket assembly 480 is disposed in and coupled with a cassette frame 106 (FIG. 7) for further processing.

Referring to FIGS. 54, 55, and 59, the cap 440 includes a sealing member 444 and at least one cap coupling member 442. In one or more embodiments, the at least one cap coupling member 442 is pivoting coupling member 446. To couple the cap 440 with the bottle 410, or release the cap 440 from the bottle 410, a user pivots the pivoting coupling member 446 to engage or release a coupling finger 450 with or from the bottle 410. In one or more embodiments, the cap 440 is coupled and/or sealed with the bottle 410. The sealing member 444 allows for the cap 440 to seal with the bottle 410. In one or more embodiments, the cap 440 is generally rectangular.

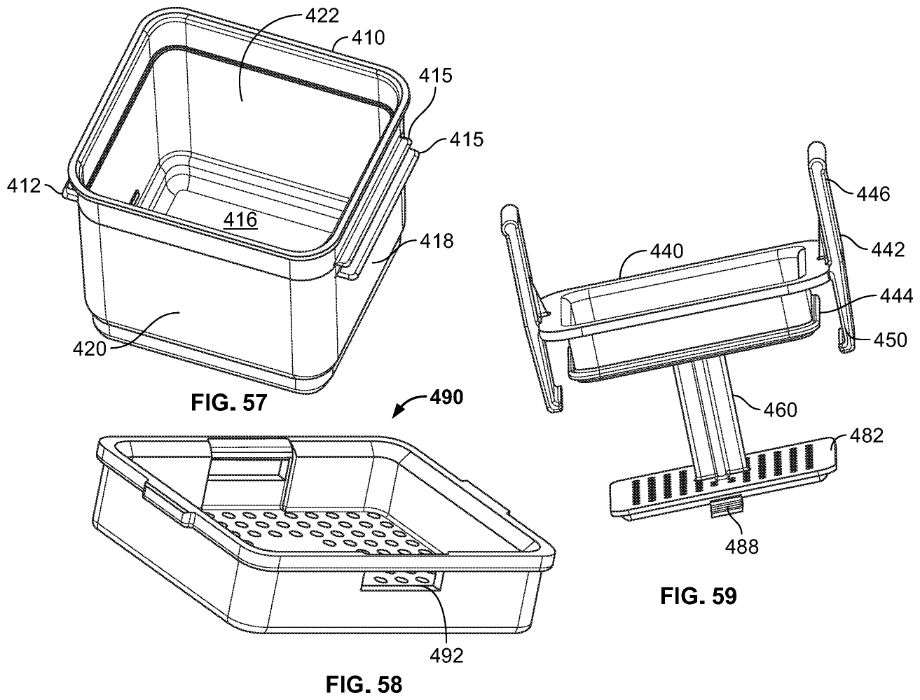

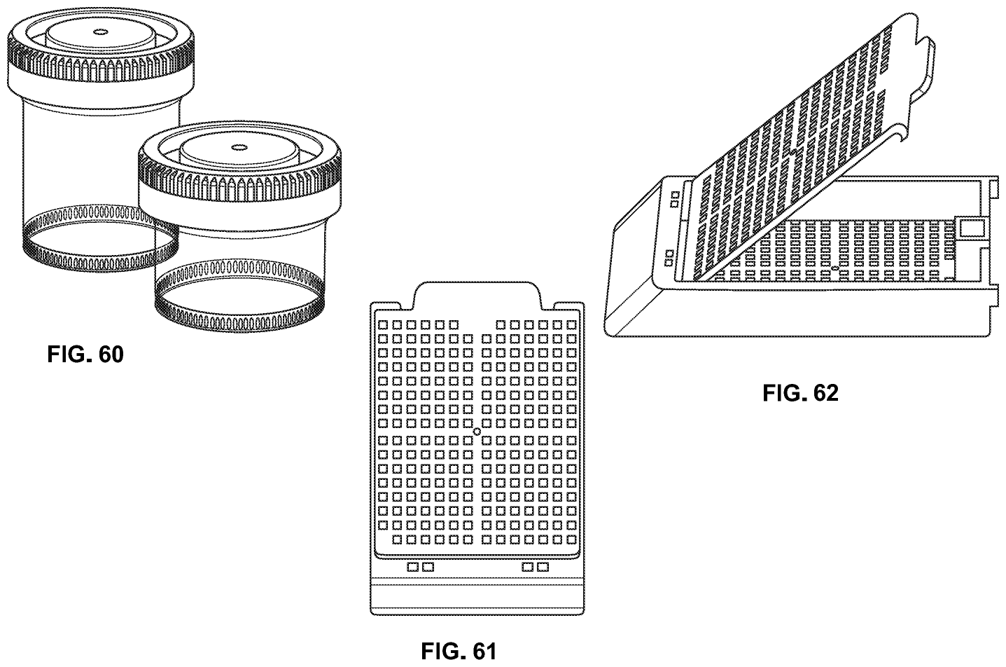

As shown in FIG. 57, the bottle 410 has an open top portion 422, and is defined in part by a bottom surface 416, a first set of side surfaces 418, and a second set of side surfaces 420. In one or more embodiments, the first set of side surfaces 418 and/or the second set of side surfaces 420 are planar. In one or more embodiments, the first set of side surfaces 418 and the second set of side surfaces 420 are perpendicular to each other.

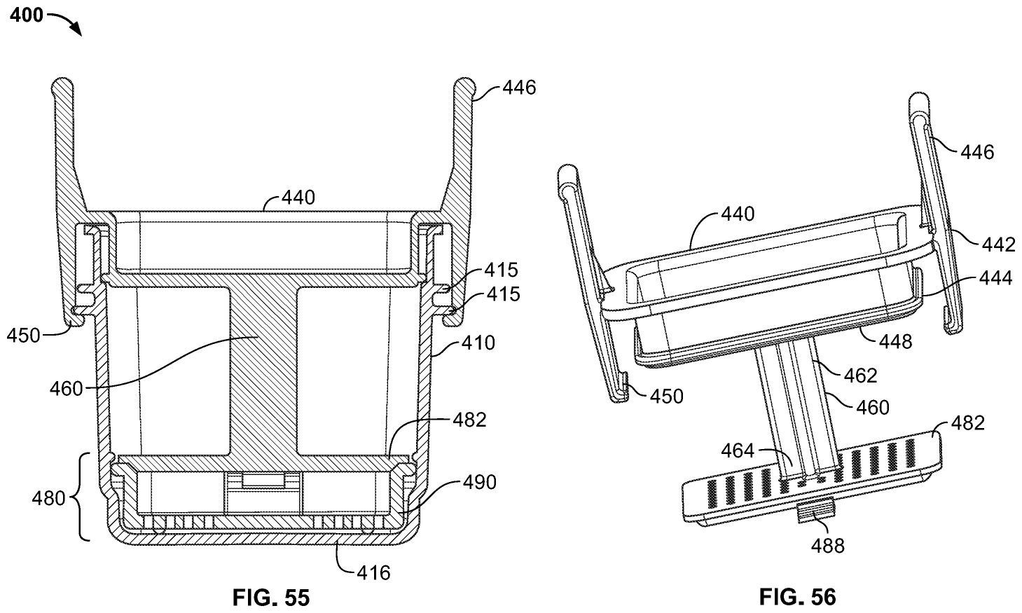

The bottle 410 includes at least one bottle coupling member 412 that allows for the cap 440 to couple with the bottle 410. In one or more embodiments, the bottle coupling member comprises a two-stage or multistage coupling portion with discrete coupling steps. In one or more embodiments, the bottle coupling member comprises two projections 415, as shown in FIGS. 53-55, 57. The projections 415 allow for the cap 440 to be coupled with the bottle 410 at two different locations relative to the bottle 410. For example, in FIG. 55 the cap 440 is seated deeper into the bottle 410 than in FIG. 54.

Disposed within the bottle 410 is an arm 460 and a basket assembly 480, as shown in FIGS. 54, 55, where the arm 460 is coupled with the cap 440. The arm 460 extends from a first end 462 to a second end 464, and the first end 462 is coupled with the inner surface 448 of the cap 440.

The basket assembly 480 includes a top basket 482 (FIGS. 56 and 59), and a bottom basket 490 (FIG. 55, 58). The top basket 482 includes one or more top basket top arm coupling members that allow for the top basket 482 to couple with the arm 460. The top basket 482 includes one or more top basket bottom coupling members 488 that allow for the top basket 482 to be coupled with the bottom basket 490. In one or more embodiments, the top basket bottom coupling members 488 include flanges that extend outward from a bottom and/or side surface of the top basket 482. The bottom basket 490 includes bottom basket coupling members 492 that allow for the bottom basket 490 to be coupled with the top basket 482. In one or more embodiments, the bottom basket coupling members 492 include one or more recesses within the bottom basket 490. In one or more embodiments, the basket assembly 480 include one or more fluid vents that allow for fluid, such as a reagent within the bottle 410, can flow freely through the basket assembly 480.

In one or more embodiments, the arm 460 and/or the top basket 482 have a first position (FIG. 54) within the bottle 410 while the cap 440 is sealed with the bottle 410. The arm 460 and/or the top basket 482 have a second position (FIG. 55) within the bottle 410 while the cap 440 is sealed with the bottle 410. In the first position, the top basket 482 is uncoupled from the bottom basket 490, as shown in FIG. 54. In the second position, the top basket 482 is coupled with the bottom basket 490, as shown in FIG. 55.

To use the tissue sample collection assembly 400, the bottom basket 490 is disposed on the bottom surface 416 of the bottle 410 and the bottle 410 is filled with a reagent. The arm 460 and top basket 482 are disposed within the bottle 410 as the cap 440 is coupled with the open top portion 422 of the bottle 410. The pivoting coupling member 446 of the cap 440 is pivoted and the cap 440 is sealed with the bottle 410 and the top basket 482 is placed in the first position, where the top basket 482 is not coupled with the bottom basket 490. At this point, the collection assembly 400 can be shipped to a clinic which harvests biopsy tissue.

When the tissue sample collection assembly 400 is at the clinic, the cap 440, arm 460, and top basket 482 (FIG. 59) are removed from the bottle 410 by pinching the pivoting coupling members 446 releasing the cap 440 from the bottle 410, and the bottom basket 490 remains in the bottom of the bottle 410. The biopsy sample can then easily be disposed within the bottom basket 490 by merely swishing the biopsy tool in the reagent and the biopsy sample falls off of the medical instrument and into the bottle 410, which is consistent with current practice in the clinic, and does not require the clinic to change current procedures. (This eliminates the extra step, as required in conventional designs of one having to open a collection basket after removing the cap, and also the biopsy tissue is more quickly placed in the reagent and has less exposure to air.) This step can be repeated multiple times as needed, until the clinic has determined that the optimal number of biopsy samples have been placed in the bottle 410. After the biopsy sample(s) has been placed in the bottle 410, some samples may have dropped into bottom basket 490, and some samples maybe floating in the reagent. The user can place the cap 440, arm 460, and top basket 482 back into the bottle 410 and capturing the biopsy sample between the top basket 482 and the bottom basket 490. The parallel position of top basket 482, the walls of the top basket 482 extending downward, and the chamfer on the inside lower edge of the walls, ensure that all biopsy samples are guided into the basket assembly 480. As the cap 440 is further inserted into the bottle 410, the top basket 482 is moved to the second position (FIG. 55), the top basket 482 is coupled with the bottom basket 490, and efficiently and securely encapsulating the tissue sample(s) within the basket assembly 480. The user releases the pivoting coupling members 446, and the coupling fingers 450 grasp the projection 415. The cap 440 is also sealed with the bottle 410, and the biopsy sample can be shipped to the lab.

When the tissue sample collection assembly 400 is received at the pathology lab, the lab can use automation to further process the biopsy sample, as shown in FIGS. 14-15, by using an automation assembly 250. The automation assembly 250 includes a fixture 258, and a robot arm 252 having a grip 254. The automation assembly 250 further includes a supply column 260, and a camera 256.

The grip 254 of the robot arm 252 picks up the tissue sample collection assembly 400 and places it in the fixture 258. In one or more embodiments, the grip 254 includes structure that allows for the tissue sample collection assembly 400 to be picked up from the side and/or from the top. For example, grip 254 includes a rounded interior portion with flanges underneath to pick up the assembly 400 from the side. The planar outer sides of the bottle mate within planar sides of the fixture, preventing the bottle from rotating or moving while the cap 440 is removed. The grip 254 is used to remove the cap 440 from the bottle 410, for example by pinching the pivoting coupling members 446. The cap 440, arm 460 and basket assembly 480 are removed from the bottle 410, and held in view of the camera 256, which documents any patient indicia and/or the number, size, shape, color and types of tissue samples within the basket assembly 480. The robot arm 252 moves the cap 440, arm 460 and basket assembly 480 over to a column of cassette frames 260, places the basket assembly 480 within the cassette frame 106. The robot arm 252 removes the arm 460 from the top basket 482 of the basket assembly 480 and returns the arm 460 and cap 440 to the bottle 410 for proper disposal. In one or more embodiments, the formalin reagent is discarded from the bottle 410 prior to placing the cap 440 back on the bottle 410. Any of the tissue sample collection assemblies herein can be used with the automation assembly 250 to further improve efficiencies of removing tissue samples from the tissue sample collection and to reduce human error.

The tissue sample collection assembly and related automation assembly allow for the biopsy tissue sample to be conveniently collected within the bottle, and efficiently allows for further automated removal of the tissue sample from the bottle at the lab and allows for further automated processing of the biopsy. This will reduce the amount of human error in removal of tissue sample and will save processing time. In addition, the tissue sample can be better documented further reducing human error.

The embodiments have been described in detail with particular reference to certain embodiments thereof, but it will be understood that variations and modifications can be made within the scope of the embodiments, especially to those skilled in the art. It should be noted that embodiments or portions thereof discussed in different portions of the description or referred to in different drawings can be combined to form additional embodiments of the present invention. The scope of the invention should, therefore, be determined with reference to the detailed descriptions, appended claims, along with the full scope of equivalents to which such claims are entitled.

1. A tissue sample collection assembly comprising:
a cap having a side wall including a cap coupling member, the cap having an inner surface;
an arm extending from a first end to a second end, the first end of the arm coupled with the inner surface of the cap;
a basket assembly including a top basket and bottom basket;
the top basket including one or more arm coupling members, the top basket coupled with the second end of the arm;
the cap coupled with a bottle, the bottle having a bottle coupling member and an open top portion, the bottle having a bottom surface, a first set of side surfaces, and a second set of side surfaces;
the basket assembly disposed within the bottle, the cap sealed with the bottle;
the arm and top basket having a first position within the bottle while the cap is sealed with the bottle;
the arm and top basket having a second position within the bottle while the cap is sealed with the bottle;
the top basket uncoupled from the bottom basket in the first position; and
the top basket coupled with the bottom basket in the second position.

2. The tissue sample collection assembly as recited in claim 1, wherein the basket assembly is rotatably coupled with the arm.

3. The tissue sample collection assembly as recited in claim 1, wherein the first set of side surfaces and the second set of side surfaces are planar.

4. The tissue sample collection assembly as recited in claim 3, wherein the first set of side surfaces and the second set of side surfaces are perpendicular to each other.

5. The tissue sample collection assembly as recited in claim 1, further comprising a guide disposed along at least one of the first set of side surfaces or the second set of side surfaces.

6. The tissue sample collection assembly as recited in claim 5, wherein the guide is a rail projecting inwardly along one or more of the first set of side surfaces or the second set of side surfaces.

7. The tissue sample collection assembly as recited in claim 5, wherein the basket assembly includes at least one basket guide member.

8. The tissue sample collection assembly as recited in claim 7, wherein the at least one basket guide member is at least one recess configured to couple with the guide of the bottle.

9. The tissue sample collection assembly as recited in claim 1, further comprising one or more top basket bottom coupling members.

10. The tissue sample collection assembly as recited in claim 1, wherein the top basket has a domed shape.

11. The tissue sample collection assembly as recited in claim 1, wherein the top basket has walls of extending downward.

12. The tissue sample collection assembly as recited in claim 1, wherein the top basket walls have a chamfer on an inside lower edge of the walls.

13. A tissue sample collection assembly comprising:
a cap having a side wall including a cap coupling member, the cap having an inner surface;
an arm extending from a first end to a second end, the first end of the arm coupled with the inner surface of the cap;
a basket assembly including a top basket and bottom basket;
the top basket including one or more arm coupling members, the top basket coupled with the second end of the arm;
the cap configured to be coupled with a bottle, the bottle having a bottle coupling member and an open top portion, the bottle having a bottom surface, a first set of side surfaces, and a second set of side surfaces;
the basket assembly disposed within the bottle, the bottom basket disposed at the bottom surface of the bottle;
the arm and top basket having a first position within the bottle when the cap is sealed with the bottle;
the arm and top basket having a second position within the bottle when the cap is sealed with the bottle;
the top basket uncoupled from the bottom basket in the first position; and
the top basket coupled with the bottom basket in the second position.

14. The tissue sample collection assembly as recited in claim 13, wherein the basket assembly is rotatably coupled with the arm.

15. The tissue sample collection assembly as recited in claim 13, wherein the first set of side surfaces and the second set of side surfaces are planar.

16. The tissue sample collection assembly as recited in any claim 15, wherein the first set of side surfaces and the second set of side surfaces are perpendicular to each other.

17-24. (canceled)

25. A method comprising:
disposing a top basket of a basket assembly within a bottle of a tissue sample collection assembly, the tissue sample collection assembly comprising a cap having a side wall including a cap coupling member, the cap having an inner surface, an arm extending from a first end to a second end, the first end of the arm coupled with the inner surface of the cap; the basket assembly including the top basket and bottom basket; the top basket including one or more arm coupling members, the top basket coupled with the second end of the arm; the cap configured to be coupled with a bottle, the bottle having a bottle coupling member and an open top portion, the bottle having a bottom surface, a first set of side surfaces, and a second set of side surfaces; the bottom basket disposed at the bottom surface of the bottle;
coupling the cap with the bottle; and
moving the top basket to a first position within the bottle and sealing the cap with the bottle, where the top basket is uncoupled from the bottom basket in the first position.

26. The method of claim 25, further comprising disposing one or more biopsy samples within the bottle prior to moving the top basket from the first position to the second position.

27. The method of claim 25, further comprising moving the top basket from the first position to the second position and coupling the top basket with the bottom basket and sealing the cap with the bottle.

28. (canceled)

29. The method of claim 25, further comprising disposing a reagent within the bottle.

30-36. (canceled)
